(19) United States
(12) Patent Application Publication
(10) Pub. No.: US 2013/0323314 A1
(43) Pub. Date: Dec. 5, 2013

(54) Novel Nanoparticles of Lactoferrin Useful for Preparing a Pharmaceutical Composition Facilitating Easy Delivery of the Drug and a Process for Preparing the Same

(71) Applicant: UNIVERSITY OF HYDERABAD, (US)

(72) Inventor: Anand Kumar Kondapi, Hyderabad (IN)

(73) Assignee: UNIVERSITY OF HYDERABAD, Hyderabad (IN)

(21) Appl. No.: 13/729,214

(22) Filed: Dec. 28, 2012

(30) Foreign Application Priority Data
Dec. 30, 2011 (IN) 4657/CHE/2011

(51) Int. Cl. A61K 38/40; A61K 45/06; A61K 9/14

(52) U.S. Cl. CPC A61K 38/40; A61K 9/14; A61K 45/06
USPC 424/499; 514/21.2; 424/130.1; 424/649

(57) ABSTRACT

Novel nanoparticles of lactoferrin useful for preparing a pharmaceutical composition facilitating easy delivery of the drug contained therein wherein the sizes are in diameter in the range of 40 to 90 nanometers.

### FIELD OF INVENTION

This invention relates to oral and intravenous formulation made of lactoferrin nanoparticles alone or lactoferrin nanoparticles loaded with pharmaceutical preparation or lactoferrin nanoparticles conjugated with pharmaceutical agent, a process for producing said nanoparticles, a pharmaceutical composition containing a drug and said nanoparticles and processes for their preparation.

The nanoparticles of lactoferrin can be directly loaded with any organic molecule or pharmaceutical preparation or protein or antibody or SiRNA or nucleic acid or other. The Composition is useful for the delivery of drugs especially anti-infective, anti-cancer drugs or neuro active agents or others.

The nanoparticles of lactoferrin can be conjugated with any reporter like Fluorescein isothiocyante or Rhodamine isothiocyante or any other (fluorescent agents) or antibodies or nucleic acids and can be used as probes for monitoring their cellular localisation. The Composition is useful for the delivery of drugs especially cancer drugs or neuro active agents or others.

Formulation containing Lactoferrin nanoparticles can be administered orally or intravenous or subcutaneous or intramuscular or other routes.

Formulation containing lactoferrin nanoparticles is safe and target-specific in localization of pharmaceutical ingredient containing in it.

### BACKGROUND OF THE INVENTION

Lactoferrin is secreted in milk, one of the iron-binding protein that possesses antimicrobial activity. Human and bovine lactoferrins have been studied in detail and their total amino acid sequences have been derived (M. W. Rey et al., Nucleic Acids Res., 18, 5288 (1990) and P. E. Mead et al., ibid., 18, 7167 (1990)). Japanese Un-examined Patent Publication No. 233226 (1989)) [Involves use of soluble lactoferrin].

It has been found lactoferrin, transferrin and ovotransferrin inhibit the infection and growth of influenza virus and cytomegalo virus (CMV) (Japanese Un-examined Patent Publication No. 233619 (1990)). Lactoferrin is effective against viruses with and without envelope. Recently, it has been confirmed that the inhibitory effect of iron-binding proteins against infection and growth of HIV is accomplished in lactoferrin containing ingredient (Japanese Patent Application No. 220635 (1992)) [Involves use of soluble lactoferrin].

A composition containing an epidermal growth factor, lactoferrins, a hydrolysate of the lactoferrins, or a mixture effects as prevention and improvement of a damage to the digestive tract, improvement of digestive tract functions deteriorating with age, and acceleration of growth and proliferation of digestive tract cells of a newborn. This mixture can be added to various food products to obtain digestive tract cells activating foods (U.S. Pat. No. 5,543,392; Tomita, et al., Aug. 6, 1996) [Involves use of soluble lactoferrin].

A composition, use and method to improve the cure of infections caused by antibiotic resistant microbial pathogens, in particular beta-lactam resistant microorganisms. Lactoferrin (LF) or Lactoferricin (LFC) can be administrated alone or in combination with antibiotic to affect growth, physiology and morphology of targeted microorganism.

Lactoferrin increase susceptibility and can reverse resistance of microorganism to antibiotics (A COMPOSITION FOR INHIBITING B-LACTAMASE IN A MICROORGRANISM; Indian Patent No. 222058, Date of Publication of Granted Patent: Jun. 18, 2008; Journal No. 29/2008).

A lactoferrin hydrolysates mixture or lactoferrin partial peptide that can be obtained by hydrolyzing lactoferrin with a hydrolytic enzyme and has an action of enhancing cytoxic activity of an antibody drug in an antibody therapy of cancer is used as active ingredient of a drug for enhancing cytotoxic activity of an antibody drug in an antibody therapy of cancer (DRUG FOR CANCER THERAPY; Indian Patent No. 231165; Date of Publication of Granted Patent: Mar. 27, 2009; Journal No. 13/2009) [Involves use of soluble lactoferrin].

A Lactoferrin composition for treating bacteremia or sepsis comprising from 0.0001% to 30% of an N-terminal Lactoferrin variant, and 0.00000001% to 60% metal chelator, wherein the metal chelator is present in a ratio of 1:10,000 to 2:1 (w/w) with the N-terminal Lactoferrin variant. (Title of the invention: “A LACTOFERRIN COMPOSITION FOR TREATING BACTEREMIA OR SEPSIS” (Indian Patent No. 245936; Date of Publication of Granted Patent: Nov. 2, 2011; Journal No. 06/2011) [Involves use of soluble lactoferrin].

A brain-targeted chemotherapeutical delivery system, doxorubicin-loaded lactoferrin-modified procationic liposome/polymersome was developed, and its therapeutic effect for glioma was evaluated (Chen et al., 2011; Pang et al., 2010) [Lactoferrin conjugated to liposomes].

Lactoferrin-modified procationic liposome (PCL), were neutral or negatively charged at physiological pH, and when they touched brain capillary endothelial cells with the help of a brain-targeting ligand, lactoferrin (Lf), they were changed into cationic liposomes (CL). Compared with the conventional liposomes and CLs, PCL and Lf-PCLs showed an improved performance in the uptake efficiency and cytotoxicity (Chen et al., 2010; Roseanu et al., 2010) [Lactoferrin conjugated to liposomes].

Polymersomes (PSs) were conjugated with Lf or Tf was shown that the uptake of Lf-PS and Tf-PS by bEnd.3 cells was time-, temperature-, and concentration-dependent (Gao et al., 2010) [Lactoferrin conjugated to polysomes].

Stabilized plasmid lipid particles (SPLPs), to which lactoferrin (LF) was coupled as a hepatocyte specific targeting ligand. Despite the efficient targeting of LF-SPLPs to hepatocytes and their capacity to transfect HepG2 and COS-7 cells in vitro, expression of a reporter gene was not detected in vivo (Weeke-Klimp et al., 2007) [Lactoferrin conjugated to liposomes].

Multi-lamellar liposomal bovine lactoferrin composed of egg yolk phosphatidylcholine and phytosterol for oral delivery. Oral pretreatment of liposomal lactoferrin exhibited more suppressive effects than did non-liposomal lactoferrin on CCl₄-induced hepatic injury in rats as well as on lipopolysaccharide-induced TNF-alpha production from mouse peripheral blood mononuclear leukocytes (Ishikado et al., 2005) [Lactoferrin linked to liposomes].

Bioadhesive tablets of bovine lactoferrin (B-LF) containing pectin, tamarind gum or carboxymethylcellulose (CMC) were developed for treatment of chronic inflammation in the oral cavity, antibacterial properties and immune regulatory functions (Takeda et al., 2007; Takahshi et al., 2007)[Lactoferrin mixed with pectin and tamrind gum or carboxymethylcellulose]

Biodegradable lactoferrin (Lf) conjugated polyethylene glycol-polylactide-polyglycolide (PEG-PLGA) nanoparticle (Lf-NP) was Constructed. Lf was thiolated and conjugated to the distal maleimide function surrounding on the pegylated nanoparticle to form Lf-NP. Intravenous administration, a near 3 fold of drug was found in the mice brain carried by Lf-NP compared to that carried by NP (Hu et al., 2011. 2009) [Lactoferrin conjugated to polymeric material].

Chitosan/alginate/calcium complex microparticles containing lactoferrin (LF) were prepared using alginate, LF, and calcium chloride at the ratio of 6:3:8 (w/w). Suppressive effect against the edema was greater in the order of microparticles LF solution control (saline)(Onishi et al., 2010) [Lactoferrin containing microparticles of chitosan/alginate/calcium complex].

FITC-labeled-lactoferrin (LF-FTC)-loaded microparticles, durable under gastrointestinal conditions, first by the combination of alginate/calcium complexation and emulsification-evaporation and next by treatment with chitosan solution (Koyama et al., 2009) [Lactoferrin loaded microparticles of alginate/calcium complex].

Chitosan microparticles loaded with LF were prepared by the w/o emulsification-solvent evaporation method using nonsonication and nonaddition of sulfate, named Ch-LF(N), showed high drug content, small particle size and spherical particle shape. Ch-LF(N) are suggested to be useful for gradual supply to topical diseased sites or for effective delivery to intestinal areas with abundant LF receptors (Onishi et al., 2007) [Lactoferrin loaded chitosan microparticles].

Lactoferrin (globular protein with antimicrobial activity) were covalently attached on the Porous silicon (PS) microparticle surface using 3-aminopropyltriethoxysilane (APTS) molecule as linker, antitumor activity is measured (Kleps et al., 2010) [Lactoferrin conjugated to Porous silicon (PS) microparticle].

Lactoferrin (Lf) was modified cationic dendrimer-based nanoparticles (NPs) were successfully exploited as a brain-targeting of Lf-modified vectors and NPs by brain capillary endothelial cells (BCECs) in a clathrin-dependent endocytosis, caveolae-mediated endocytosis, and macropinocytosis (Huang et al., 2009) [Lactoferrin conjugated to cationic dendrimer-based nanoparticles (NPs].

Liquid filled nano- and micro-particles (LFNPS/LFMPS) were prepared using solid adsorbents such as porous silicon dioxide (Sylysia 550), carbon nanotubes (CNTs), carbon nanohorns, fullerene, charcoal and bamboo charcoal. Surfactants such as a saturated polyglycolysed C8-C18 glyceride (Gelucire 44/14), PEG-8 capryl/caprylic acid glycerides (Labrasol) and polyoxyethylene hydrogenated castor oil derivative (HCO-60) were used as an absorption enhancer at 50 mg/kg along with casein/lactoferrin as enzyme inhibitors (Venkatesan et al., 2005) [Lactoferrin absorbed into nano- and micro-particles (LFNPS/LFMPS)].

Superparamagnetic iron oxide nanoparticles with specific shape and size coupled lactoferrin binds to cell membrane (Gupta and Curtis, 2004) [Lactoferrin conjugated to Superparamagnetic iron oxide nanoparticles].

Present invention involves novel nanoparticles composed on lactoferrin alone or lactoferrin nanoparticles containing a pharmacologically active ingredient, which is entirely different from (a) soluble lactoferrin, (b) liposomal preparation containing lactoferrin in free or conjugated form, (c) microparticles conjugated with lactoferrin, and (d) nanoparticles conjugated with lactoferrin. Novel lactoferrin nanoparticles in the present invention do not involved any conjugation, thus retain natural character of the protein in nanopartcile form. In addition, absence of conjugation of drug allow the efficient loading of drug as conjugation is not limiting step in drug concentration, further release of drug is direct without having any de-conjugation or de-coupling related rate-limiting in drug release. Thus the novel nano particles of lactoferrin provided by the present invention would help providing a non-toxic delivery system.

### OBJECTS OF THE INVENTION

An object of the present invention is to provide a novel nanoparticles of lactoferrin which are useful for preparing a pharmaceutical composition facilitating easy delivery of the drug contained in the composition;

Another object of the present invention is to provide novel nanoparticles of lactoferrin having sizes, in diameter, in the range of 40 to 60 nanometers which are useful for preparing a pharmaceutical composition facilitating easy delivery of the drug contained in the composition;

Yet another object of the present invention is to provide a process for the preparation of the novel nanoparticles of lactoferrin which are useful for preparing a pharmaceutical composition facilitating easy delivery of the drug contained in the composition;

Still another object of the present invention is to provide novel pharmaceutical composition containing a drug and the novel nanoparticles of lactoferrin which is useful for preparing a pharmaceutical composition facilitating easy delivery of the drug contained in the composition;

Yet another object of the present invention is to provide a novel pharmaceutical composition in which lactoferrin is readily available after the delivery of the drug contained in the composition.

Still another object of the present invention is to provide a novel pharmaceutical composition containing a drug and the novel nanoparticles of lactoferrin which do not induce any cytotoxicity.

Another object the present invention is to provide a process for the preparation of novel pharmaceutical composition containing a drug and the novel nanoparticles of lactoferrin which is useful for the preparation of a pharmaceutical composition facilitating easy delivery of the drug contained in the composition

Still another object the present invention is to provide a process for the preparation of novel pharmaceutical composition containing a drug and the novel nanoparticles of lactoferrin which is useful for target delivery of the drug without employing chemical conjugation of the drug and lactoferrin;

The use of nanoparticles of lactoferrin in combination with the drug can enhance the stability of the binding of the drug to lactoferrin. Such a combination also enhances the delivery of the drug into the cells and reduces the competition cellular iron binding with the drug. The resulting composition does not also cause any toxicity to the patients;

The nanoparticles of lactoferrin present in the composition can bind to lactoferrin receptors and drug enters all lactoferrin receptor expressing cells, the extent of localization of particles (along with the drug) is high in lactoferrin receptor over expressing cells such as cancer cells, brain and other infections and diseases where iron transport is very active. Hence, the drug can be selectively targeted to cancer, brain and other infections/diseases through these novel nano particles of lactoferrin;

To the best of our knowledge nanoparticles of lactoferrin is not hitherto known and we are the first to prepare such nanoparticles and our above mentioned findings are disclosed for the first time. Hence the invention disclosed in this application is novel.

### BRIEF DESCRIPTION OF THE INVENTION

According to this invention there is provided a novel nanoparticles of lactoferrin useful for preparing a pharmaceutical composition facilitating easy delivery of the drug contained therein wherein the sizes are in diameter in the range of 40 to 60 nanometers.

In accordance with this invention there also provided a process for the preparation of the novel nanoparticles of lactoferrin which are useful for preparing a pharmaceutical composition facilitating easy delivery of the drug which comprises:
i) dissolving lactoferrin in a solvent selected from, Phosphate buffer saline or Tris buffer saline or water;
ii) dispersing the solution obtained in step (i) in an oil at a temperature in the range of 4° C. to 30° C.,
iii) sonicating the solution obtained in step (ii) in a sonicator using a probe by passing the pulse for 2 second pulse and 2 seconds gap with continuously for 15 minutes;
iv) Freezing the sonicated solution obtained in step (iii) for at least 15 minutes below (minus) −20° C. followed by incubation between 4 to 30° C. for 2 to 8 hours;
v) Separating the nanoparticles formed by centrifugation for 10-30 minutes,
vi) Discarding the supernatant and washing the nanoparticles repeatedly using ice cold diethyl ether or any oil miscible solvent and
vii) Dispersing the resultant nanoparticles of lactoferrin in saline or a buffer or water.

Accordingly to another embodiment of the present invention provides novel nanoparticles of lactoferrin useful as microbicide;

According to another embodiment of the present invention there is provided novel nanoparticles of lactoferrin having sizes in diameter in the range of 40 to 60 nanometers useful for the preparation of a pharmaceutical composition facilitating easy delivery of the drug contained in the composition;

In an embodiment the invention ratio of the solution obtained and the oil used in step (ii) is at least 1:35. The oil which can be used in step (ii) may be selected from olive oil, vegetable oil, sesame oil, castor oil, peanut oil, sunflower oil, rice bran oil, safflower oil, mustard oil.

The pulse amplitude of sonication used in step (iii) ranges from 50 to 80% power and the probe used is 0.375 inches diameter solid titanium tip Cat No. 0-120-0009 on Ultrasonic Homogenizer Model 300V/T of Bioloics Inc., USA or equivalent.

In the step (iv) the sonicated solution is freezed immediately. The centrifugation in step (v) may be carried out at least 5000 rpm.

The dispersion of the particles in step (v) may be done immediately after washing in ice cold ether or any oil miscible solvent

To make the formulation for administration in vivo particles were dissolved in mannitol and lyophilized. Lyophilized powder can disperse particles directly.

According to another embodiment of the present invention there is provided a novel pharmaceutical composition containing a drug and the novel nanoparticles of lactoferrin facilitating easy delivery of the drug contained in the composition

According to yet another embodiment of the present invention there is provided a novel pharmaceutical composition containing a drug and the novel nanoparticles of lactoferrin in which lactoferrin is readily available after the delivery of the drug

According to still another embodiment of the present invention there is provided a novel pharmaceutical composition containing a drug and the novel nanoparticles of lactoferrin, which do not induce any cytotoxicity.

According to still another embodiment of the present invention there is provided a novel pharmaceutical composition containing a drug and the novel nanoparticles of lactoferrin suspended in water or phosphate buffered saline or saline or any buffer as oral as well as intravenous formulation.

According to still another embodiment of the present invention there is provided a novel pharmaceutical composition containing a drug and the novel nanoparticles of lactoferrin administered orally and intravenous route enhances pharmacokintic profile and bioavailability of drug

According to still another embodiment of the present invention there is provided a novel pharmaceutical composition containing a drug and the novel nanoparticles of lactoferrin administered orally and intravenous route improves safety of drug.

According to still another embodiment of the present invention there is provided a novel pharmaceutical composition containing the novel nanoparticles of lactoferrin alone with anti-HIV activity.

Though any drugs can be used in the pharmaceutical composition of the present invention, in particular, the drugs selected from antivirals, antibiotics, anti cancer agents, neuroactive agents, proteins, antibodies, RNA, DNA and the like may be used.

Preferably the antibiotics used may be selected from Cefuroxime, cholroquine and the like.

Preferably the anti-cancer agents used may be selected from etoposide, gemcitabine, paclitaxel, irinotecan, mitomycin, bleomycin, doxorubicin, carboplatin, oxaliplatin, cisplatin, nitrosourea,

Another embodiment of the present invention is to provide a process for the preparation of novel pharmaceutical composition containing a drug and the novel nanoparticles of lactoferrin which is useful for preparing a pharmaceutical composition facilitating easy delivery of the drug which comprises.

i) dissolving lactoferrin in a solvent selected from, Phosphate buffer saline or Tris buffer saline or water

ii) dispersing the solution obtained in step (i) in an oil at a temperature in the range of 4° C. to 30° C.,

iii) sonicating the solution obtained in step (ii) in a sonicator using a probe by passing the pulse for 2 second pulse and 2 seconds gap with continuously for 15 minutes.

iv) freezing the sonicated solution obtained in step (iii) for at least 15 minutes below (minus) −20° C. followed by incubation between 4 to 30° C. for 2 to 8 hours.

v) separating the nanoparticles formed by centrifugation for 10-30 minutes,

vi) discarding the supernatant and washing the nanoparticles repeatedly using ice cold diethyl ether or any oil miscible solvent and

vii) Dispersing the resultant nanoparticles of lactoferrin in saline or a buffer or water.

In an embodiment the invention ratio of the solution obtained and the oil used in step (ii) is at least 1:35. The oil which can be used in step (ii) may be selected from olive oil, vegetable oil, sesame oil, castor oil, peanut oil, sunflower oil, rice bran oil, safflower oil, mustard oil.

The pulse amplitude of sonication used in step (iii) ranges from 50 to 80% power and the probe used is 0.375 inches diameter solid titanium tip Cat No. 0-120-0009 on Ultrasonic Homogenizer Model 300V/T of Bioloics Inc., USA or equivalent.

In the step (iv) the sonicated solution is freezed immediately. The centrifugation in step (v) may be carried out at least 5000 rpm.

The dispersion of the particles in step (v) may be done immediately after washing in ice cold ether or any oil miscible solvent

To make the formulation for administration in vivo particals were dissolved in mannitol and lyophilized. Lyophilized powder can disperse particles directly.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIGS. 1A and 1B show SEM, TEM, and AFM characterization of lactoferrin nanoparticles prepared by sol-oil chemistry.

FIG. 2. shows sub cellular localization of Rhodamine123 (Green)-tagged lactoferrin nanoparticles treated with fluorescently endosome labeled (Blue).

FIGS. 3A and 3B show SEM, TEM, and AFM characterization of doxorubicin-loaded lactoferrin nanoparticles prepared by sol-oil chemistry.

FIG. 4 shows inhibition of the proliferation of SKNSH neuroblastoma and SupT1 T cell lymphoma cell lines doxorubicin-loaded nanoparticles.

FIG. 5 shows tissue distribution of sol doxo (A) and lacto doxo (B) doxorubicin-loaded lactoferrin nanoparticles in brain, liver, heart, kidney, spleen, lungs, bone marrow and blood following oral administration. Protein was precipitated in 30% AgNO3 and drug was extracted in methanol.

FIG. 6 shows H&E staining of doxorubicin-loaded lactoferrin nanoparticles under bright field microscopy following oral administration. Tissues were collected, fixed in 4% PFA and embedded in paraffin stained with hematoxyline and eosin for slide preparation and gelatine coated slides.

FIG. 7 shows staining of doxorubicin-loaded lactoferrin nanoparticles under Leica laser confocal microscopy following oral administration. Tissues were collected, fixed in 4% PFA and embedded in paraffin. Drug (red) and protein (green) detection is shown.

FIG. 8 shows a safety profile of doxorubicin-loaded lactoferrin nanoparticles following oral administration. Safety analysis shows doxorubicin alone is cardiotoxic, nano form of doxo shows no toxicity to heart.

FIG. 9 shows tissue distribution of lacto doxo (A) and sol doxo (B) doxorubicin-loaded lactoferrin nanoparticles following intravenous administration in brain, liver, heart, kidney, spleen, lungs, bone marrow and blood following oral administration. Protein was precipitated in 30% AgNO3 and drug was extracted in methanol.

FIG. 10 shows H&E staining of doxorubicin-loaded lactoferrin nanoparticles under bright field microscopy following intravenous administration. Tissues were collected, fixed in 4% PFA and embedded in paraffin stained with hematoxyline and eosin for slide preparation and gelatine coated slides.

FIG. 11 shows staining of doxorubicin-loaded lactoferrin nanoparticles under Leica laser confocal microscopy following intravenous administration. Tissues were collected, fixed in 4% PFA and embedded in paraffin. Drug (red) and protein (green) detection is shown.

FIG. 12 shows a safety profile of doxorubicin-loaded lactoferrin nanoparticles following intravenous administration. Safety analysis shows doxorubicin alone is cardiotoxic, nano form of doxo shows no toxicity to heart.

FIG. 13 shows tissue distribution of lacto doxo (A) and sol doxo (B) doxorubicin-loaded lactoferrin nanoparticles following intramuscular administration in brain, liver, heart, kidney, spleen, lungs, bone marrow and blood following oral administration. Protein was precipitated in 30% AgNO3 and drug was extracted in methanol.

FIG. 14 shows H&E staining of doxorubicin-loaded lactoferrin nanoparticles under bright field microscopy following intramuscular administration. Tissues were collected, fixed in 4% PFA and embedded in paraffin stained with hematoxyline and eosin for slide preparation and gelatine coated slides.

FIG. 15 shows staining of doxorubicin-loaded lactoferrin nanoparticles under Leica laser confocal microscopy following intramuscular administration. Tissues were collected, fixed in 4% PFA and embedded in paraffin. Drug (red) and protein (green) detection is shown.

FIG. 16 shows a safety profile of doxorubicin-loaded lactoferrin nanoparticles following intramuscular administration. Safety analysis shows doxorubicin alone is cardiotoxic, nano form of doxo shows no toxicity to heart.

FIG. 17 shows tissue distribution of sol doxo (A) and lacto doxo (B) doxorubicin-loaded lactoferrin nanoparticles following subcutaneous administration in brain, liver, heart, kidney, spleen, lungs, bone marrow and blood following oral administration. Protein was precipitated in 30% AgNO3 and drug was extracted in methanol.

FIG. 18 shows H&E staining of doxorubicin-loaded lactoferrin nanoparticles under bright field microscopy following subcutaneous administration. Tissues were collected, fixed in 4% PFA and embedded in paraffin stained with hematoxyline and eosin for slide preparation and gelatine coated slides.

FIG. 19 shows staining of doxorubicin-loaded lactoferrin nanoparticles under Leica laser confocal microscopy following subcutaneous administration. Tissues were collected, fixed in 4% PFA and embedded in paraffin. Drug (red) and protein (green) detection is shown.

FIG. 20 shows a safety profile of doxorubicin-loaded lactoferrin nanoparticles following subcutaneous administration. Safety analysis shows doxorubicin alone is cardiotoxic, nano form of doxo shows no toxicity to heart.

FIG. 21 shows hepatocellular carcinoma in control, sol doxo treated, and lacto doxo nano treated liver nodules. Carcinoma was induced by adding 100 mg/L of diethyl nitrosamine in drinking water for eight weeks. Cancer was confirmed by visualizing the liver nodules.

FIG. 22 shows H&E staining of liver nodules following no drug or 4 μg/g of drug equivalent of lacto doxo nano or sol doxo through oral administration Hepatocellular carcinoma was induced by adding 100 mg/L of diethyl nitrosamine (DENA) in drinking water for eight weeks. On the last day of DENA administration animals received 4 μg/g of drug equivalent. Hepatocellular carcinoma was reduced when treated with lacto doxo nano.

FIG. 23 shows a safety profile showing that oral doxorubicin alone is cardiotoxic, nano form of doxo shows no toxicity to heart.

FIG. 24 shows concentration of sol doxo and lacto doxo in liver, kidney, heart, and spleen in rats with hepatocellular carcinoma following oral administration.

FIG. 25 shows hepatocellular carcinoma in control, sol doxo treated, and lacto doxo nano treated liver nodules. Carcinoma was induced by adding 100 mg/L of diethyl nitrosamine in drinking water for eight weeks. Cancer was confirmed by visualizing the liver nodules.

FIG. 26 shows H&E staining of liver nodules following no drug or 4 μg/g of drug equivalent of lacto doxo nano or sol doxo through intravenous administration Hepatocellular carcinoma was induced by adding 100 mg/L of diethyl nitrosamine (DENA) in drinking water for eight weeks. On the last day of DENA administration animals received 4 μg/g of drug equivalent. Hepatocellular carcinoma was reduced when treated with lacto doxo nano.

FIG. 27 shows a safety profile showing that intravenous doxorubicin alone is cardiotoxic, nano form of doxo show no toxicity to heart.

FIG. 28 shows concentration of sol doxo and lacto doxo in liver, kidney, heart, and spleen in rats with hepatocellular carcinoma following intravenous administration.

FIG. 29 shows FeSEM characterization of etoposide-loaded lactoferrin nanoparticles prepared by sol-oil chemistry.

FIG. 30 shows inhibition of the proliferation of COLO 205 cell line by etoposide-loaded lactoferrin nanoparticles.

FIG. 31 shows FeSEM characterization of carboplatin-loaded lactoferrin nanoparticles prepared by sol-oil chemistry.

FIG. 32 shows inhibition of the proliferation of Y79 retinblastoma cell line by carboplatin-loaded lactoferrin nanoparticles.

FIG. 33 shows localization of doxorubicin-loaded lactoferrin nanoparticles and lactoferrin nanoparticles following administration to Y79 cell line and incubated to indicated time points and observed under laser confocal microscopy. Nanoparticles localized in cytosol of Y79 cell line and drug was localized in nucleus.

FIG. 34 shows kinetics of drug localization of doxorubicin-loaded lactoferrin nanoparticles and soluble doxo following administration to Y79 retinoblastoma cell line.

FIG. 35 shows comparative localization of doxorubicin-loaded apotransferrin nanoparticle and soluble doxorubicin in the retina of rat eye at different time points. Apotransferrin labeled with Rhodamine(green), Doxorubicin (red), OS: outer Segment, ONL: outer nuclear layer, INL: inner nuclear layer, INL:inner plexiform layer.

FIG. 36 shows FeSEM characterization of paclitaxol-loaded lactoferrin nanoparticles prepared by sol-oil chemistry.

FIG. 37 shows inhibition of the proliferation of supT1 T cell lymphoma cell line by paclitaxol-loaded lactoferrin nanoparticles.

FIG. 38 shows FeSEM characterization of 5-fluorouracil-loaded lactoferrin nanoparticles prepared by sol-oil chemistry.

FIG. 39 shows inhibition of proliferation of supT1 T cell lymphoma cell line by 5-fluorouracil-loaded lactoferrin nanoparticles.

FIG. 40 shows FeSEM characterization of cyclophosphomide-loaded lactoferrin nanoparticles prepared by sol-oil chemistry.

FIG. 41 shows inhibition of proliferation of COLO 205 cell line by cyclophosphomide-loaded lactoferrin nanoparticles.

FIG. 42 shows FeSEM characterization of gemcitabine-loaded lactoferrin nanoparticles prepared by sol-oil chemistry.

FIG. 43 shows inhibition of proliferation of COLO 205 cell line by gemcitabine-loaded lactoferrin nanoparticles.

FIG. 44 shows FeSEM characterization of cisplatin-loaded lactoferrin nanoparticles prepared by sol-oil chemistry.

FIG. 45 shows inhibition of proliferation of COLO 205 cell line by cisplatin-loaded lactoferrin nanoparticles.

FIG. 46 shows FeSEM characterization of methotrexate-loaded lactoferrin nanoparticles prepared by sol-oil chemistry.

FIG. 47 shows inhibition of proliferation of supT1 T cell lymphoma cell line by methotrexate-loaded lactoferrin nanoparticles.

FIG. 48 shows FeSEM characterization of cytarabine-loaded lactoferrin nanoparticles prepared by sol-oil chemistry.

FIG. 49 shows inhibition of proliferation of supT1 T cell lymphoma cell line by cytarabine-loaded lactoferrin nanoparticles.

FIG. 50 shows FeSEM characterization of AZT-loaded lactoferrin nanoparticles prepared by sol-oil chemistry.

FIG. 51 shows FeSEM characterization of T20-loaded lactoferrin nanoparticles prepared by sol-oil chemistry.

FIG. 52 shows anti HIV-1 activity of lacto nano, lacto AZT and lacto T20. Soluble drugs are the controls.

### DETAILED DESCRIPTION OF THE INVENTION

The details of the Process for producing the composition

Lactoferrin is very stable protein and is present in high concentrations in human milk, we isolated from goat and bovine milk. It can be easily isolated (Garcia-Montoya et al., 1996; Onishi H., 2011). According to the process of the present invention, no chemical modification of the drug is required. The drug can be directly incorporated into the nanoparticles of lactoferrin/holo lactoferrin through adsorption, absorption and occlusion during the particle formation. The resulting composition can be used to for the delivery of the drug contained in the composition to treat diseases due to cancer, brain and other infectious and diseases in which lactoferrin receptor is over expressed.

These nanoparticles lactoferrin/holo lactoferrin can bind to lactoferrin receptor is an ˜110 kDa trimeric (˜37 kDa subunits), glycosylated protein in intestine, heart, liver, spleen, thymus, spermatozoa and skeletal muscle (Suzuki et al, 2001, 2004, 2005; Kawakami et al., 1991, Wang et al., 2011) and the composition of the present invention enters all lactoferrin receptor expressing cells. The extent of localization of particles (along with drug) is high in lactoferrin receptor expressing cells such as human bronchial epithelial cells (Elfinger et al., 2007), cancer cells (Tuccari et al., 1999; Legrand et al., 1998; Birgens et al., 1984; Gibbons et al., 2011; Tuccari and Barresi, 2011), brain (Huang et al., 2007), neurodegenerative diseases such as Parkinson's disease, Alzheimer's disease, Huntington's disease and amyotrophic lateral sclerosis (Hirsch, 2009; Faucheux et al., 1995; Qian and Wang, 1998) and other microbial infections and diseases where iron transport is very active (Beddek and Schryvers, 2010; Furano and Campagnari, 2004; Ekins et al., 2004; Anderson et al., 2003; Yu and Schryvers, 2002; Ogunnariwo and Schryvers, 2001; Dhaenens et al., 1997; Bonnah et al., 1995; Lee, 1992; Vogel et al., 1997). Furthermore, lactoferrin itself show virucidal activity (Valenti and Antonini, 2005) against DNA- and RNA-viruses, including rotavirus, respiratory syncytial virus, herpes viruses (Jenssen, 2005; Seganti et al., 2005; van der Strate et al., 2001), HIV (Berkhout et al., 2004) and HCMV (Florisa e tal., 2003). Hence, active ingredient can be selectively targeted to cancer, brain and other infections/diseases demanding high iron metabolism (Iyer and Lonnerdal, 1993) through these particles.

The process of the lactoferrin/lactoferrin nano particle entry involves binding of the particle to lactoferrin receptors followed by cellular entry. During the entry the protein in the particle is undergoes molecular changes leading to the release of the active ingredient into the cytosol. The lactoferrin/holo lactoferrin is converted to soluble from and the soluble lactoferrin/holo lactoferrin undergoes recycling. Thus it will support the iron transport and associated metabolic activity. Since the lactoferrin/holo lactoferrin is recycled, it does not possess any toxicity unlike in other delivery systems tested for lactoferrin receptor specific targeting rather the present delivery system will be supportive to cell. As the drug is released in cytosol, the particles are not required to enter sub-organelles for release of active ingredient.

After the release of the drug rest of lactoferrin/holo lactoferrin is available for recycling for iron transport for the cell, thus increase iron intake and associated metabolic activation and associated active ingredient action. Thus the risk of iron depletion and activation resistance mechanism can be avoided by using the composition of the present invention. If use is made of soluble lactoferrin/holo lactoferrin method, the drug requires iron or iron like center for binding to the active site of lactoferrin/holo lactoferrin and soluble drug bound to lactoferrin/holo lactoferrin may undergo competition to free soluble iron in vivo. The extent of the release of the drug depends on the stability of the drug interaction with the active site of lactoferrin/holo lactoferrin compared to that with soluble iron. In present invention the nanoparticles of lactoferrin/holo lactoferrin present in the composition is insoluble, hence, it cannot participate in soluble lactoferrin and free iron equilibria, hence cannot be destabilized in vivo.

The invention is described in detail in the examples given below which are given to illustrated the invention only and therefore should not be constructed it limit the scope of the invention

### Example 1

### Preparation of Lactoferrin Nanoparticle

25 mg of a solution of lactoferrin was prepared in 100 μl of phosphate buffer saline and incubated on ice for 5 min. Cold lactoferrin solution is slowly added in steps of 0.01 ml to 15 ml of olive oil at 4° C. with continuous dispersion by gentle manual vortexing.

The particle formation of lactoferrin in oil phase was initiated by sonication using ranges from 50 to 80% power and the probe used is 0.375 inches diameter solid titanium tip Cat No. 0-120-0009 on Ultrasonic Homogenizer Model 300V/T of Bioloics Inc., USA and by passing the pulse for 2 second pulse and 2 seconds gap with continuously for 15 minutes.

After sonication of the olive oil containing lactoferrin, the resulting mixture was immediately frozen in liquid nitrogen at −196° C. for 10 min. Then it was transferred to ice and incubated for 4 hours. The Particles formed were pelleted by spinning at 5000 rpm for 10 minutes. The pellets of the composition of nano particles of lactoferrin obtained is decanted and was washed twice using 15 ml of ice cold diethyl ether. The pellets are immediately dispersed thoroughly by manual vortexing in 1 ml of phosphate buffered saline. The dispersed pellets were passed through the 0.2 micron filter and the filtrate obtained contained lactoferrin nano particles. These nano particles can be stored for one week at 4° C. and can be stored for more than a month at below 0° C. temperature.
24

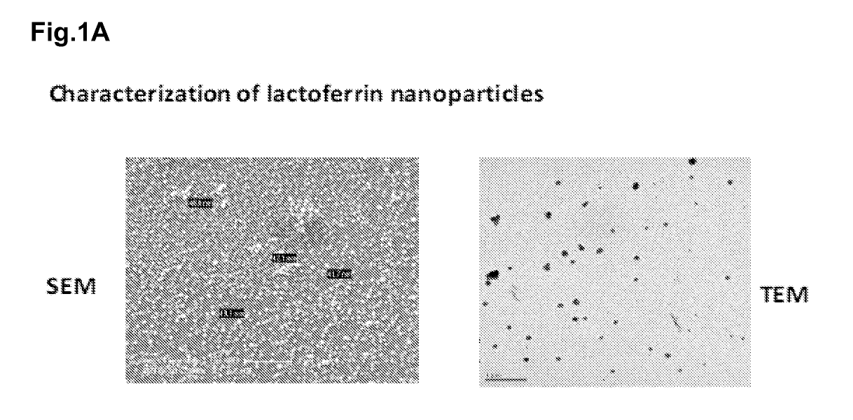

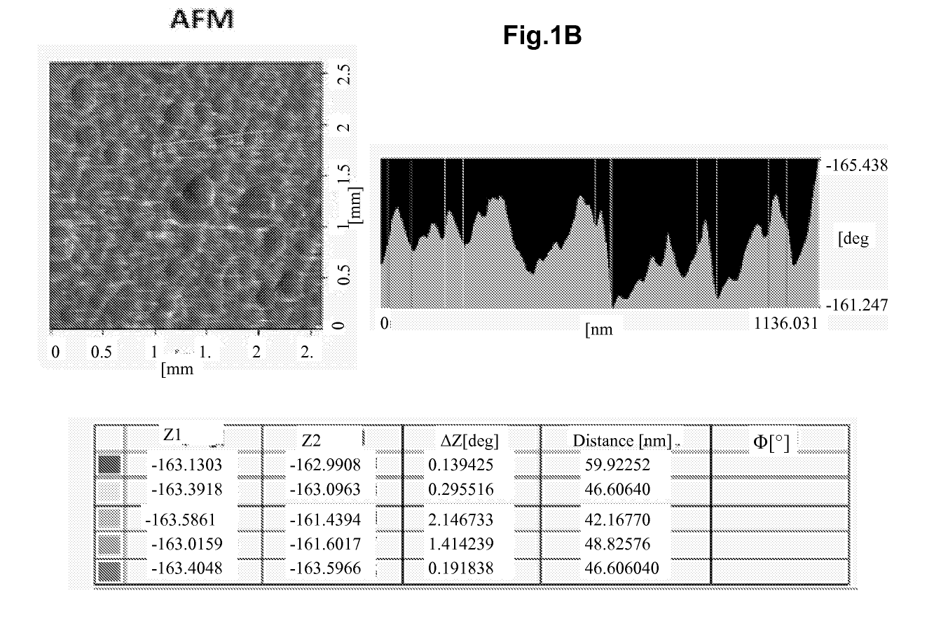

The nanoparticles of lactoferrin-are characterized as 40 to 50 nano meters as shown in FIG. 1 of the drawing accompanying this specification. These nano particles are stable in water and get dissociated in acidic conditions (less than pH 3).

### Example 2

### Preparation of Conjugated Lactoferrin Nano Particle

25 mg of a solution of lactoferrin-conjugated with Rodamine¹²³ was prepared in 100 μl of phosphate buffer saline and incubated on ice for 5 min. Cold lactoferrin solution is slowly added in steps of 0.01 ml to 15 ml of olive oil at 4° C. with continuous dispersion by gentle manual vortexing.

The particle formation of lactoferrin in oil phase was initiated by sonication using ranges from 50 to 80% power and the probe used is 0.375 inches diameter solid titanium tip Cat No. 0-120-0009 on Ultrasonic Homogenizer Model 300V/T of Bioloics Inc., USA and by passing the pulse for 2 second pulse and 2 seconds gap with continuously for 15 minutes. After sonication of the olive oil containing lactoferrin, the resulting mixture was immediately frozen in liquid nitrogen at −196° C. for 10 min. Then it was transferred to ice and incubated for 4 hours. The Particles formed were pelleted by spinning at 5000 rpm for 10 minutes. The pellets of the composition of nano particles of lactoferrin obtained is decanted and was washed twice using 15 ml of ice cold diethyl ether. The pellets are immediately dispersed thoroughly by manual vortexing in 1 ml of phosphate buffered saline. The dispersed pellets were passed through the 0.2 micron filter and the filtrate obtained contained lactoferrin nano particles. These nano particles can be stored for one week at 4° C. and can be stored for more than a month at below 0° C. temperature.

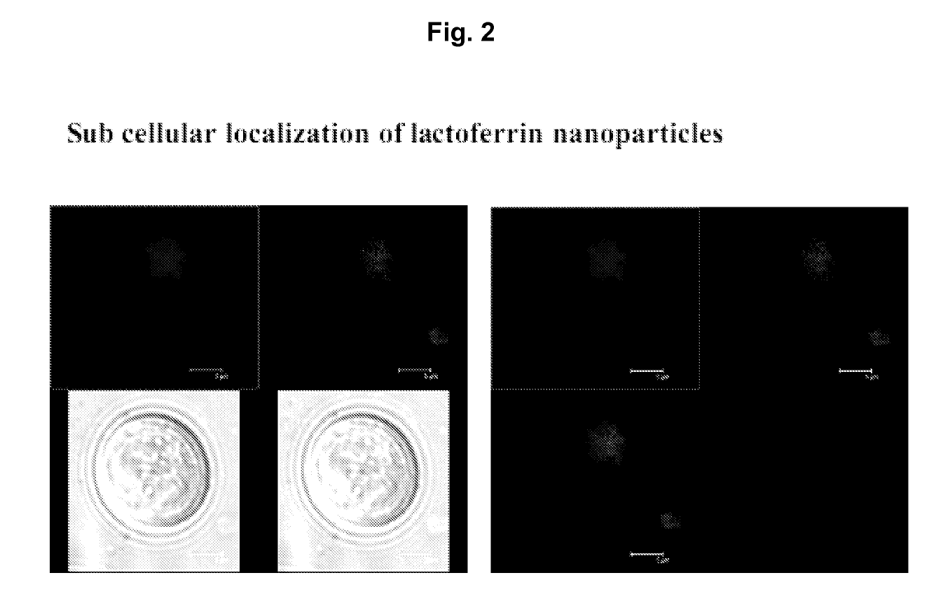

The nanoparticles of lactoferrin are characterized as 40 to 50 nano meters. These nanoparticles are stable in water and get dissociated in acidic conditions (less than pH 3). Fluorescence analysis for the presence of lactoferrin Protein was done using Rh¹²³ (Rhodamine¹²³). The Rh¹²³ labeled protein was used to prepare lactoferrin protein particles and such particles were used to monitor the localization of drug in cells and organelles using the laser confocal microscope with the Rh¹²³ excitation (511) and emission (534). The results of these studies show that the lactoferrin nanoparticles are localized in cytosol of the cells as shown in FIG. 2.

### Example-3

### Composition Containing Doxorubicin Hydrochloride- and Lactoferrin Nano Particles

25 mg of lactoferrin in 100 μl of phosphate buffer saline was slowly mixed with the 5.3 mg of Doxorubicin hydrochloride in 100 μl of phosphate buffer saline and incubated in ice for 5 min. the mixture of lactoferrin and the drug was slowly added in steps of 0.01 ml to 15 ml of olive oil at 4° C. with continuous dispersion by gentle manual vortexing.

The particle formation of lactoferrin-Doxorubicin hydrochloride in oil phase was initiated by sonication using ranges from 50 to 80% power and the probe used is 0.375 inches diameter solid titanium tip Cat No. 0-120-0009 on Ultrasonic Homogenizer Model 300V/T of Bioloics Inc., USA and by passing the pulse for 2 second pulse and 2 seconds gap with continuously for 15 minutes. After sonication of the olive oil containing lactoferrin-Doxorubicin hydrochloride the resulting mixture was immediately frozen in liquid nitrogen at −196° C. for 10 min. Then it was transferred to ice and incubated for 4 hours. The Particles formed were pelleted by spinning at 6000 rpm for 10 minutes. The pellets of the composition of nano particles of lactoferrin and Doxorubicin hydrochloride obtained is decanted and was washed twice using 15 ml of ice cold diethyl ether. The pellets are immediately dispersed thoroughly by manual vortexing in 1 ml of phosphate buffered saline. The dispersed pellets were passed through the 0.2 micron filter and the filtrate obtained contained lactoferrin-Doxorubicin hydrochloride nano particles. These nano particles can be stored for one week at 4° C. and can be stored for more than a month at below 0° C. temperature.

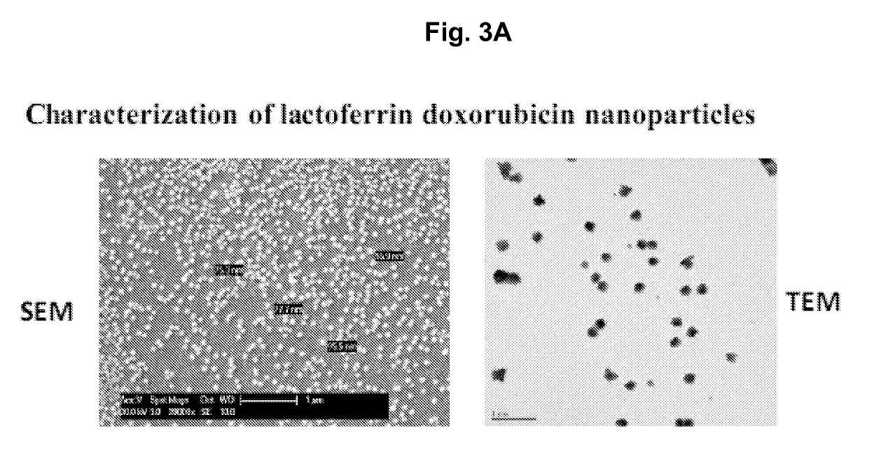

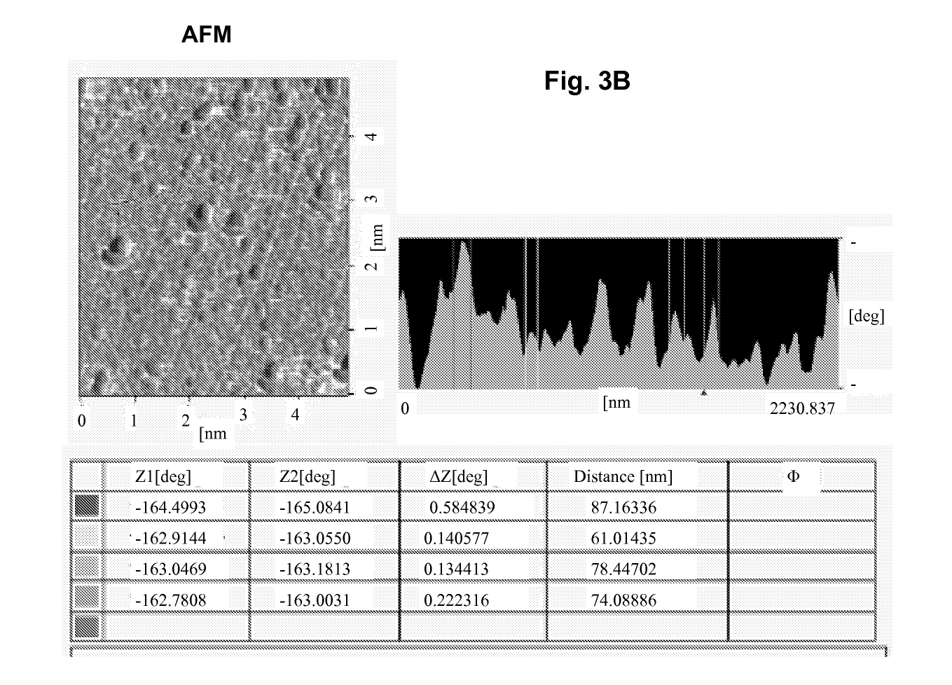

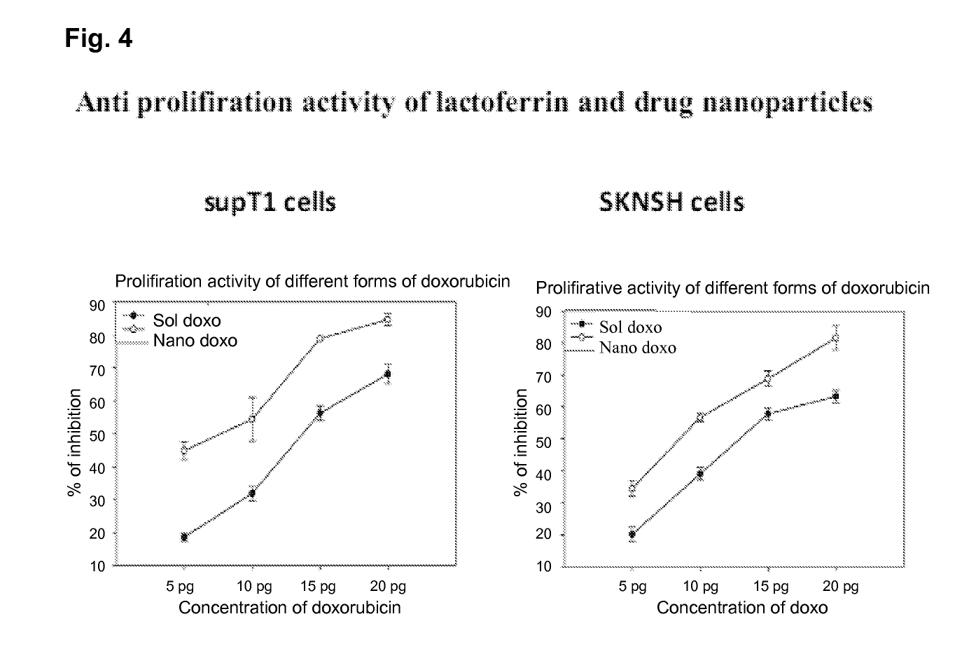

The nano particles of lactoferrin-Doxorubicin hydrochloride were characterized as 68 to 75 nano meters as shown in FIG. 3. These nano particles were stable in water and got dissociated in acidic conditions (less than pH 3). These particles inhibited proliferation of SKNSH neuroblastoma, SupT1 T cell lymphoma cell line as shown in FIG. 4.

### Example-4

### Oral Formulation Containing Doxorubicin Hydrochloride Loaded Lactoferrin Nano Particles

25 mg of lactoferrin in 100 μl of phosphate buffer saline was slowly mixed with the 5.3 mg of Doxorubicin hydrochloride in 100 μl of phosphate buffer saline and incubated in ice for 5 min. the mixture of lactoferrin and the drug was slowly added in steps of 0.01 ml to 15 ml of olive oil at 4° C. with continuous dispersion by gentle manual vortexing.

The particle formation of lactoferrin-Doxorubicin hydrochloride in oil phase was initiated by sonication using ranges from 50 to 80% power and the probe used is 0.375 inches diameter solid titanium tip Cat No. 0-120-0009 on Ultrasonic Homogenizer Model 300V/T of Bioloics Inc., USA and by passing the pulse for 2 second pulse and 2 seconds gap with continuously for 15 minutes. After sonication of the olive oil containing lactoferrin-Doxorubicin hydrochloride the resulting mixture was immediately frozen in liquid nitrogen at −196° C. for 10 min. Then it was transferred to ice and incubated for 4 hours. The Particles formed were pelleted by spinning at 5000 rpm for 10 minutes. The pellets of the composition of nano particles of lactoferrin and Doxorubicin hydrochloride obtained is decanted and was washed twice using 15 ml of ice cold diethyl ether. The pellets are immediately dispersed thoroughly by manual vortexing in 1 ml of phosphate buffered saline. The dispersed pellets were passed through the 0.2 micron filter and the filtrate obtained contained lactoferrin-Doxorubicin hydrochloride nano particles. These nano particles can be stored for one week at 4° C. and can be stored for more than a month at below 0° C. temperature. These nanoparticles are lyophilized in the presence of mannitol. Lyophilized nanoparticles formulation can be dispersed freely in water or when administered orally.

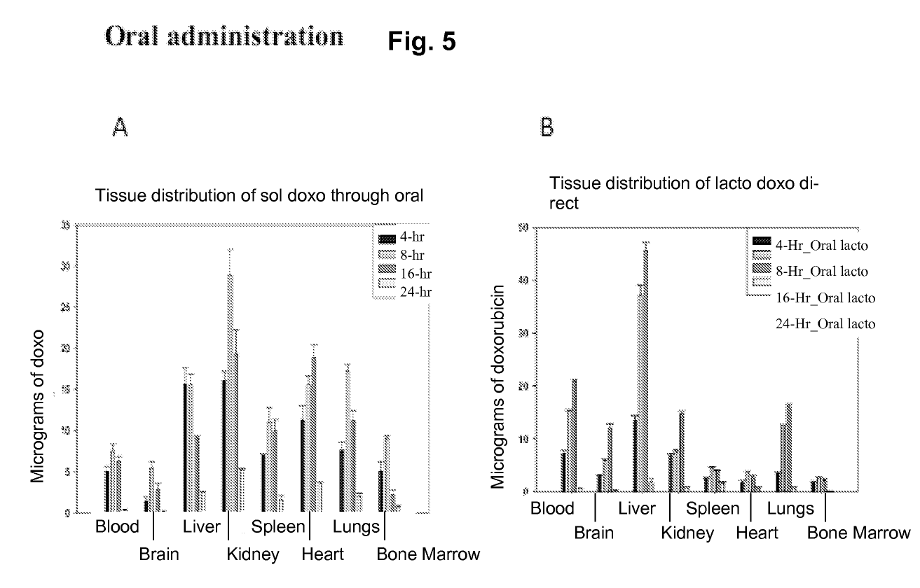

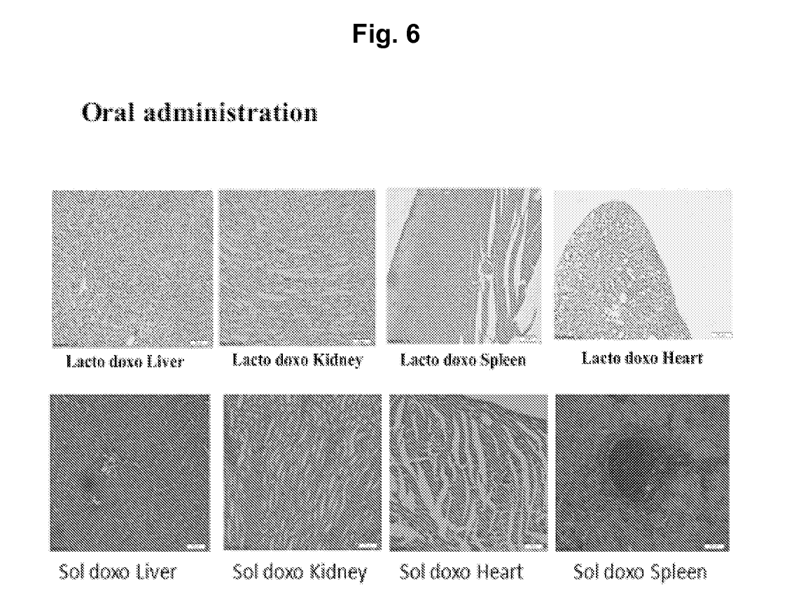

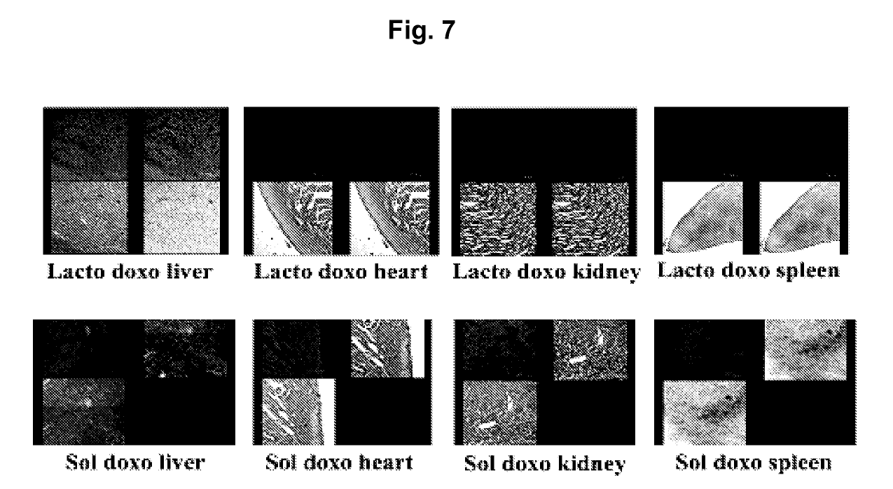

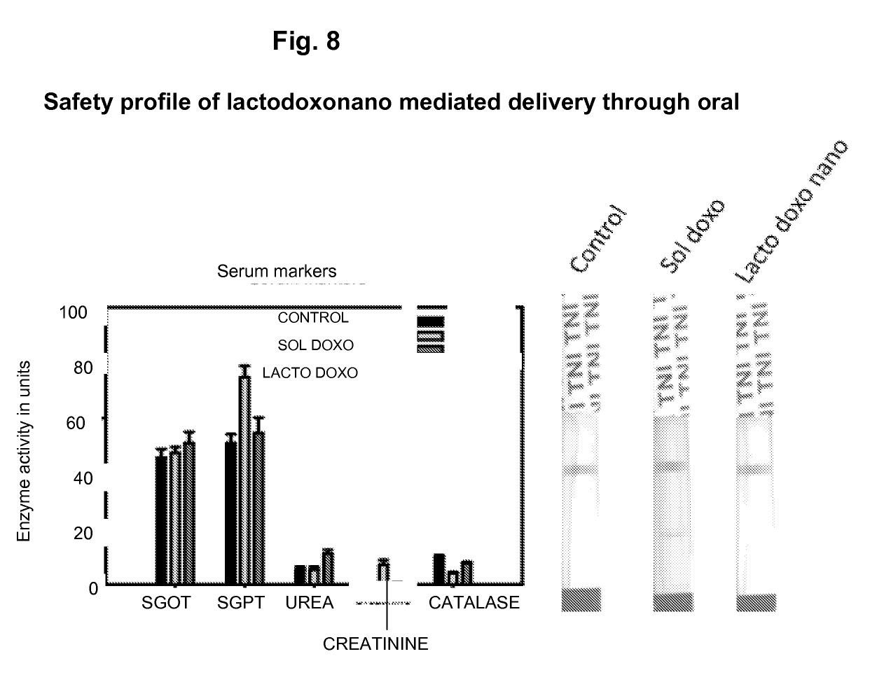

The nano particles of lactoferrin-doxorubicin were characterized as ˜68 nano meters. These nano particles are stable in water and get dissociated in acidic conditions (less than pH 3). It shows oral biovailability and less toxicity then doxorubicin. Lacto doxo nanoparticles show very less toxicity to heart. Tissues like Brain, liver, heart, Kidney, Spleen, lungs, bone marrow and blood are collected, protein was precipitated in 30% AgNO₃ and drug was extracted in methanol. Drug was estimated with spectrophotometer show tissue distribution FIG. 5. Small piece of collected tissues are fixed in 4% PFA and embedded in paraffin stained with hematoxyline and eosin for slide preparation and gelatine coated slides. H & E coated slides observed under bright field microscope FIG. 6. These gelatine coated slides examined under Leica laser confocal microscope for drug and protein detection FIG. 7. Pharmacokinetics parameters shown in Table-1. Safety analysis shows doxorubicin alone is cardiotoxic, nano form of doxo show no toxicity to heart FIG. 8.

TABLE 1

| | | | Lactodoxo | | | | LactoDoxo | | Lactodoxo |
|---|---|---|---|---|---|---|---|---|---|
| | Units | Doxo Blood | Blood | | Units | Doxo Liver | Liver | Doxo Heart | Heart |
| AUC | (h) * (μg/μL) | 105.24 | 210.234 | AUC | (h) * (μg/g) | 207.816 | 537.561 | 248.048 | 52.0521 |
| AUMC | (h)\hat{ }2 * (μg/μL) | 1227.63 | 2411.27 | AUMC | (h)\hat{ }2 * (μg/g) | 2379.51 | 6500.26 | 3211.86 | 843.387 |
| MRT last | Hr | | 7.96253 | MRT | Hr | 7.42846 | 5.32133 | 8.9708 | 9.07309 |
| Cmax | μg/μL | 8.25 | 15.32 | last | | | | | |
| Tmax | Hr | 8 | 8 | Cmax | μg/g | 17.4 | 36.32 | 17.25 | 6.12 |
| t½ | Hr | 3.74142 | 4.52386 | Tmax | Hr | 8 | 8 | 16 | 16 |
| | | | | t½ | Hr | 6.09886 | 5.25985 | 7.53301 | 5.16275 |

| | | | LactoDoxo | | LactoDoxo |
|---|---|---|---|---|---|
| | Units | Doxo Kidney | Kidney | Doxo Spleen | Spleen |
| AUC | (h) * (μg/g) | 362.639 | 45.052 | 190.928 | 67.5284 |
| AUMC | (h)\hat{ }2 * (μg/g) | 4321.31 | 105.62 | 2616.55 | 1286.41 |
| MRT last | Hr | 8.2078 | 9.58008 | 9.2978 | 9.09508 |
| Cmax | μg/g | 32.9 | 14.2 | 11.105 | 4.19 |
| Tmax | Hr | 8 | 16 | 8 | 16 |
| t½ | Hr | 6.46976 | 6.48169 | 5.89392 | 6.48744 |

Pharmacokinetics parameters
AUC: The integral of the concentration-time curve (after a single dose or in steady state).
T½: The time required for the concentration of the drug to reach half of its original value.
Cₘₐₓ: The peak plasma concentration of a drug after oral administration.
Tₘₐₓ: Time to reach Cₘₐₓ

### Example 5

### Intravenous Formulation Containing Doxorubicin Hydrochloride Loaded Lactoferrin Nano Particles

25 mg of lactoferrin in 100 μl of phosphate buffer saline was slowly mixed with the 5.3 mg of Doxorubicin hydrochloride in 100 μl of phosphate buffer saline and incubated in ice for 5 min. the mixture of lactoferrin and the drug was slowly added in steps of 0.01 ml to 15 ml of olive oil at 4° C. with continuous dispersion by gentle manual vortexing. The particle formation of lactoferrin-Doxorubicin hydrochloride in oil phase was initiated by sonication using ranges from 50 to 80% power and the probe used is 0.375 inches diameter solid titanium tip Cat No. 0-120-0009 on Ultrasonic Homogenizer Model 300V/T of Bioloics Inc., USA and by passing the pulse for 2 second pulse and 2 seconds gap with continuously for 15 minutes. After sonication of the olive oil containing lactoferrin-Doxorubicin hydrochloride the resulting mixture was immediately frozen in liquid nitrogen at −196° C. for 10 min. Then it was transferred to ice and incubated for 4 hours. The Particles formed were pelleted by spinning at 5000 rpm for 10 minutes. The pellets of the composition of nano particles of lactoferrin and Doxorubicin hydrochloride obtained is decanted and was washed twice using 15 ml of ice cold diethyl ether. The pellets are immediately dispersed thoroughly by manual vortexing in 1 ml of phosphate buffered saline. The dispersed pellets were passed through the 0.2 micron filter and the filtrate obtained contained lactoferrin-Doxorubicin hydrochloride nano particles. These nano particles can be stored for one week at 4° C. and can be stored for more than a month at below 0° C. temperature. These nanoparticles are lyophilized in the presence of mannitol. Lyophilized nanoparticles formulation can be dispersed freely in water and administered intravenous route.

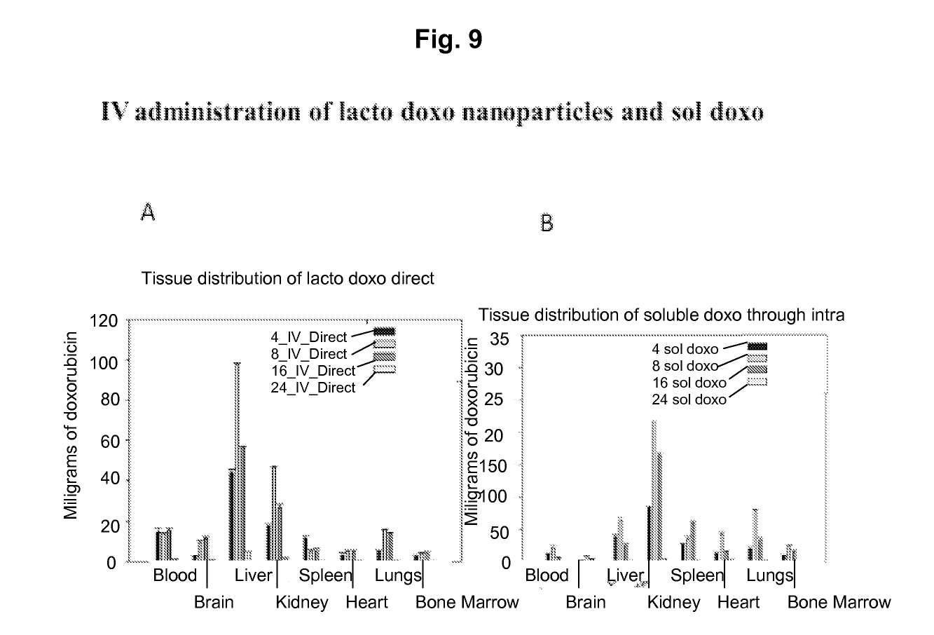

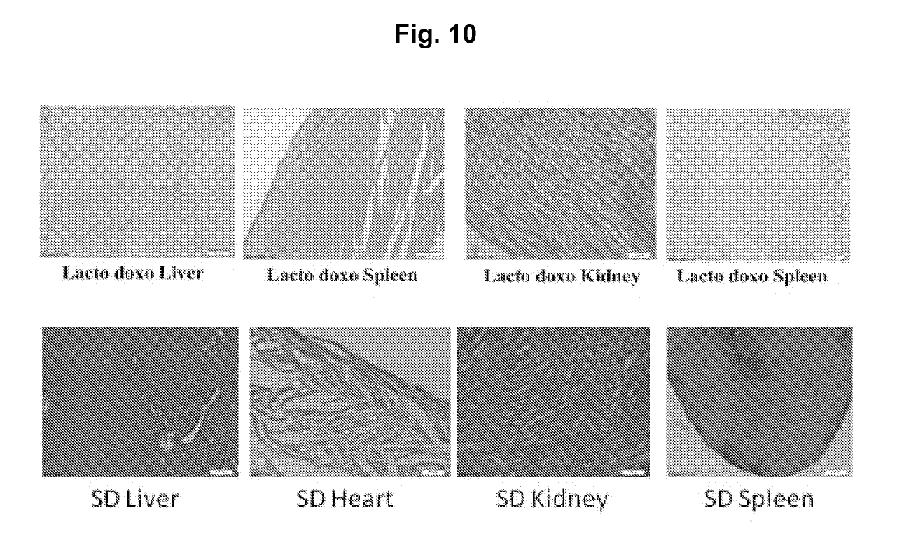

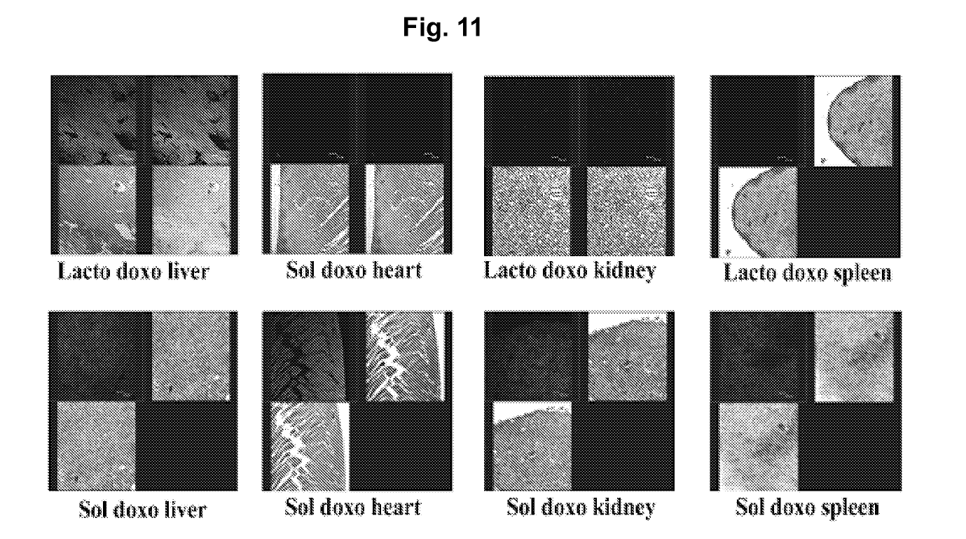

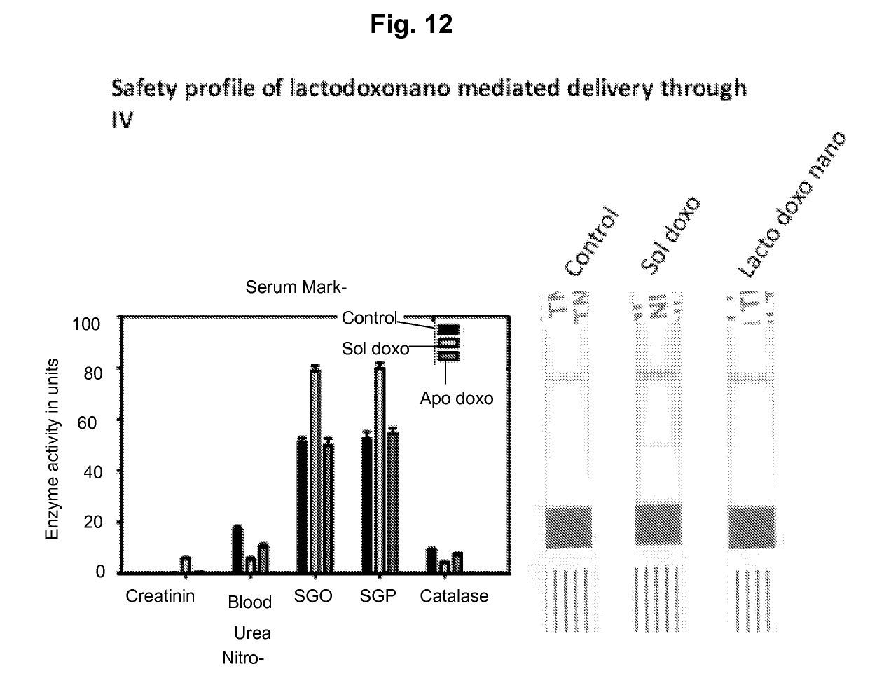

The nano particles of lactoferrin-doxorubicin were characterized as ˜68 nano meters. These nano particles are stable in water and get dissociated in acidic conditions (less than pH 3). Lacto-doxo nanoparticles are administered through intravenous and sacrifice the animal at indicated time points. Tissues like Brain, liver, heart, Kidney, Spleen, lungs, bone marrow and blood are collected, protein was precipitated in 30% AgNO₃ and drug was extracted in methanol. Drug was estimated with spectrophotmeter show tissue distribution FIG. 9. Small piece of collected tissues are fixed in 4% PFA and embedded in paraffin stained with hematoxyline and eosin for slide preparation and gelatine coated slides. H & E coated slides observed under bright field microscope FIG. 10. These gelatine coated slides examined under Leica laser confocal microscope for drug and protein detection FIG. 11. Pharmacokinetics parameters shown in table-2. Safety analysis shows doxorubicin alone is cardiotoxic, nano form of doxo show no toxicity to heart FIG. 12.

TABLE 2

| | | | Lactodoxo | | | | LactoDoxo | | Lactodoxo |
|---|---|---|---|---|---|---|---|---|---|
| | Units | Doxo Blood | Blood | | Units | Doxo Liver | Liver | Doxo Heart | Heart |
| AUC | (h) * (μg/μL) | 206.564 | 223.535 | AUC | (h) * (μg/g) | 713.776 | 644.915 | 445.749 | 66.33 |
| AUMC | (h)\hat{ }2 * (μg/μL) | 1977.42 | 2717.94 | AUMC | (h)\hat{ }2 * | 7360.72 | 8364.55 | 5360.59 | 932.66 |
| | | | | | (μg/g) | | | | |
| Cmax | μg/μL | 22.2867 | 15.2667 | Cmax | Hr | 63.9967 | 38.6833 | 43.35 | 4.95 |
| Tmax | Hr | 8 | 8 | Tmax | μg/g | 8 | 8 | 8 | 16 |
| t½ | Hr | 2.73838 | 4.49983 | t½ | Hr | 3.43151 | 5.10182 | 4.44442 | 5.16 |
| MRT last Hr | | | | MRT last | Hr | | | | |

| | Units | Doxo Kidney | LactoDoxo Kidney | Doxo Spleen | LactoDoxo Spleen |
|---|---|---|---|---|---|
| AUC | (h) * (μg/g) | 3428 | 169.3 | 445.3 | 65.18 |
| AUMC | (h)\hat{ }2 * (μg/g) | 37367 | 2583 | 4793 | 957.4 |
| Cmax | Hr | 353.7 | 12.32 | 30.55 | 3.988 |
| Tmax | μg/g | 8 | 16 | 8 | 8 |
| t½ | Hr | 2.63 | 6.482 | 3.833 | 6.487 |
| MRT last | Hr | | | | |

Pharmacokinetics parameters
AUC: The integral of the concentration-time curve (after a single dose or in steady state).
T½: The time required for the concentration of the drug to reach half of its original value.
Cₘₐₓ: The peak plasma concentration of a drug after oral administration.
Tₘₐₓ: Time to reach Cₘₐₓ

### Example 6

### Intramuscular Formulation Containing Doxorubicin Hydrochloride Loaded Lactoferrin Nano Particles

25 mg of lactoferrin in 100 μl of phosphate buffer saline was slowly mixed with the 5.3 mg of Doxorubicin hydrochloride in 100 μl of phosphate buffer saline and incubated in ice for 5 min. the mixture of lactoferrin and the drug was slowly added in steps of 0.01 ml to 15 ml of olive oil at 4° C. with continuous dispersion by gentle manual vortexing.

The particle formation of lactoferrin-Doxorubicin hydrochloride in oil phase was initiated by sonication using ranges from 50 to 80% power and the probe used is 0.375 inches diameter solid titanium tip Cat No. 0-120-0009 on Ultrasonic Homogenizer Model 300V/T of Bioloics Inc., USA and by passing the pulse for 2 second pulse and 2 seconds gap with continuously for 15 minutes. After sonication of the olive oil containing lactoferrin-Doxorubicin hydrochloride the resulting mixture was immediately frozen in liquid nitrogen at −196° C. for 10 min. Then it was transferred to ice and incubated for 4 hours. The Particles formed were pelleted by spinning at 5000 rpm for 10 minutes. The pellets of the composition of nano particles of lactoferrin and Doxorubicin hydrochloride obtained is decanted and was washed twice using 15 ml of ice cold diethyl ether. The pellets are immediately dispersed thoroughly by manual vortexing in 1 ml of phosphate buffered saline. The dispersed pellets were passed through the 0.2 micron filter and the filtrate obtained contained lactoferrin-Doxorubicin hydrochloride nano particles. These nano particles can be stored for one week at 4° C. and can be stored for more than a month at below 0° C. temperature. These nanoparticles are lyophilized in the presence of mannitol. Lyophilized nanoparticles formulation can be dispersed freely in water and administered intramuscular route.

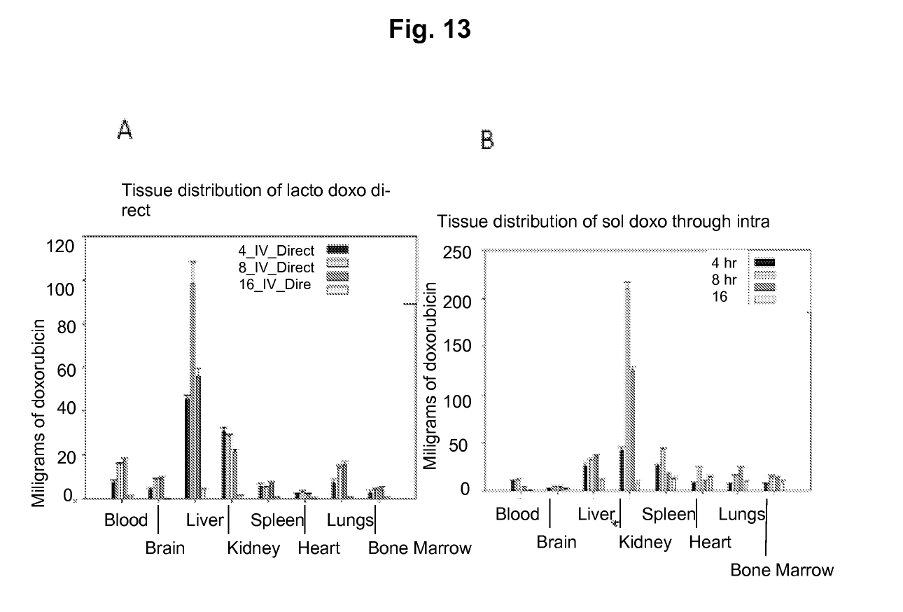

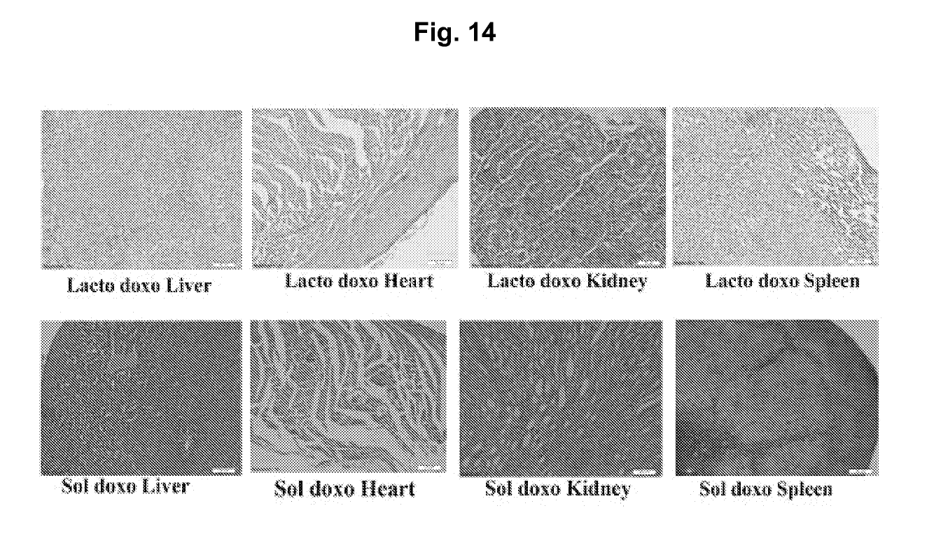

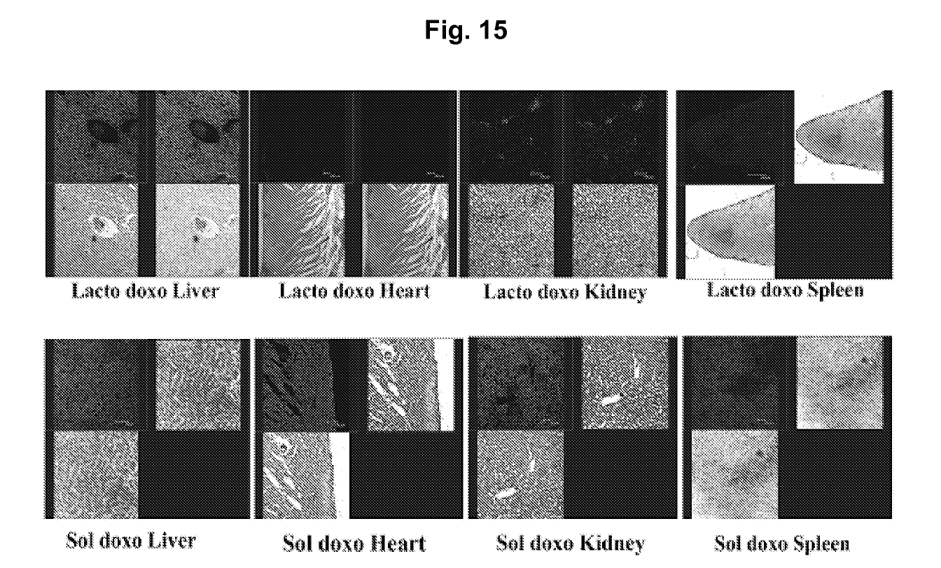

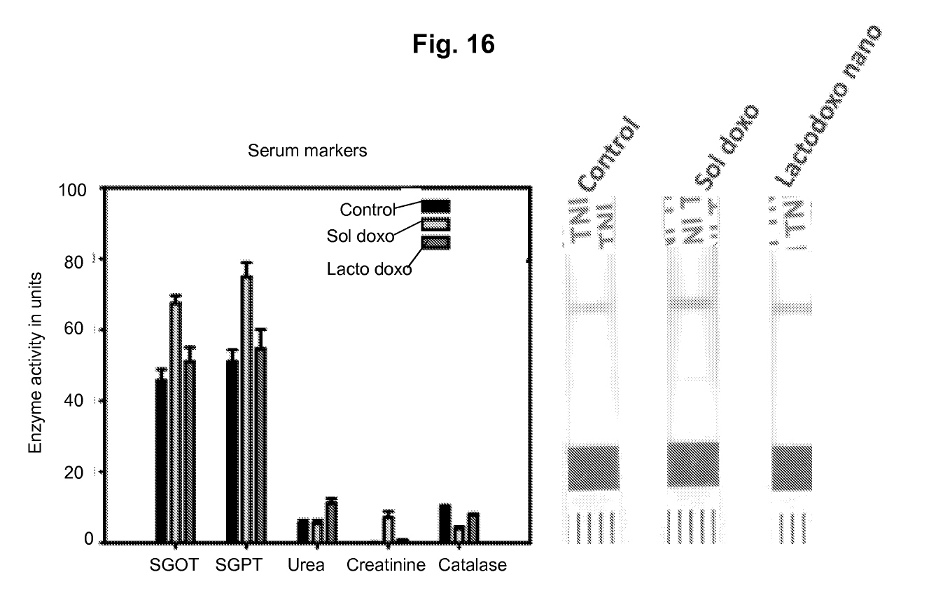

The nano particles of lactoferrin-doxorubicin were characterized as ˜68 nano meters. These nano particles are stable in water and get dissociated in acidic conditions (less than pH 3). Lacto-doxo nanoparticles are administered through intramascular and sacrifice the animal at indicated time points. Tissues like Brain, liver, heart, Kidney, Spleen, lungs, bone marrow and blood are collected, protein was precipitated in 30% AgNO₃ and drug was extracted in methanol. Drug was estimated with spectrophotometer show tissue distribution FIG. 13. Small piece of collected tissues are fixed in 4% PFA and embedded in paraffin stained with hematoxyline and eosin for slide preparation and gelatine coated slides. H & E coated slides observed under bright field microscope FIG. 14. These gelatine coated slides examined under Leica laser confocal microscope for drug and protein detection FIG. 15. Pharmacokinetics parameters shown in Table-3. Safety analysis shows doxorubicin alone is cardiotoxic, nano form of doxo show no toxicity to heart FIG. 16.

TABLE 3

| | | Sol doxo | Lactodoxo | | | Sol doxo | Lacto Doxo | Sol doxo | Lactodoxo |
|---|---|---|---|---|---|---|---|---|---|
| | Units | Blood | Blood | | Units | Liver | Liver | Heart | Heart |
| AUC | (h) * (μg/μL) | 185.4 | 237.58 | AUC | (h) * (μg/g) | 751.01 | 1363.6 | 510.54 | 63.655 |
| AUMC | (h)\hat{ }2 * (μg/μL) | 1581.8 | 2474.8 | AUMC | (h)\hat{ }2 * (μg/g) | 10278 | 12491 | 12046 | 777.97 |
| Cmax | μg/μL | 11.032 | 16.272 | Cmax | Hr | 36.488 | 96.505 | 22.953 | 5.385 |
| Tmax | Hr | 8 | 8 | Tmax | μg/g | 16 | 8 | 8 | 8 |
| t½ | Hr | 4.466 | 3.5891 | t½ | Hr | 4.9231 | 2.798 | 13.27 | 2.9743 |
| MRT | Hr | 8.5317 | 10.417 | MRT last | Hr | 13.685 | 9.1604 | 23.595 | 12.222 |
| last | | | | | | | | | |

| | Units | Sol doxo Kidney | LactoDoxo Kidney | Sol doxo Spleen | Lacto Doxo Spleen |
|---|---|---|---|---|---|
| AUC | (h) * (μg/g) | 2580.5 | 441.89 | 899.9 | 14.825 |
| AUMC | (h)\hat{ }2 * (μg/g) | 26548 | 4311.6 | 19234 | 170.4 |
| Cmax | Hr | 210.24 | 22.68 | 42.633 | 1.0833 |
| Tmax | μg/g | 8 | 8 | 8 | 16 |
| t½ | Hr | 2.0438 | 3.6651 | 13.602 | 1.8227 |
| MRT last | Hr | 10.288 | 9.7572 | 21.374 | 11.494 |

Pharmacokinetics parameters
AUC: The integral of the concentration-time curve (after a single dose or in steady state).
T½: The time required for the concentration of the drug to reach half of its original value.
Cₘₐₓ: The peak plasma concentration of a drug after oral administration.
Tₘₐₓ: Time to reach Cₘₐₓ

### Example-7

### Subcutaneous Formulation Containing Doxorubicin Hydrochloride Loaded Lactoferrin Nano Particles

25 mg of lactoferrin in 100 μl of phosphate buffer saline was slowly mixed with the 5.3 mg of Doxorubicin hydrochloride in 100 μl of phosphate buffer saline and incubated in ice for 5 min. the mixture of lactoferrin and the drug was slowly added in steps of 0.01 ml to 15 ml of olive oil at 4° C. with continuous dispersion by gentle manual vortexing.

The particle formation of lactoferrin-Doxorubicin hydrochloride in oil phase was initiated by sonication using ranges from 50 to 80% power and the probe used is 0.375 inches diameter solid titanium tip Cat No. 0-120-0009 on Ultrasonic Homogenizer Model 300V/T of Bioloics Inc., USA and by passing the pulse for 2 second pulse and 2 seconds gap with continuously for 15 minutes. After sonication of the olive oil containing lactoferrin-Doxorubicin hydrochloride the resulting mixture was immediately frozen in liquid nitrogen at −196° C. for 10 min. Then it was transferred to ice and incubated for 4 hours. The Particles formed were pelleted by spinning at 5000 rpm for 10 minutes. The pellets of the composition of nano particles of lactoferrin and Doxorubicin hydrochloride obtained is decanted and was washed twice using 15 ml of ice cold diethyl ether. The pellets are immediately dispersed thoroughly by manual vortexing in 1 ml of phosphate buffered saline. The dispersed pellets were passed through the 0.2 micron filter and the filtrate obtained contained lactoferrin-Doxorubicin hydrochloride nano particles. These nano particles can be stored for one week at 4° C. and can be stored for more than a month at below 0° C. temperature. These nanoparticles are lyophilized in the presence of mannitol. Lyophilized nanoparticles formulation can be dispersed freely in water and administered subcutaneous route.

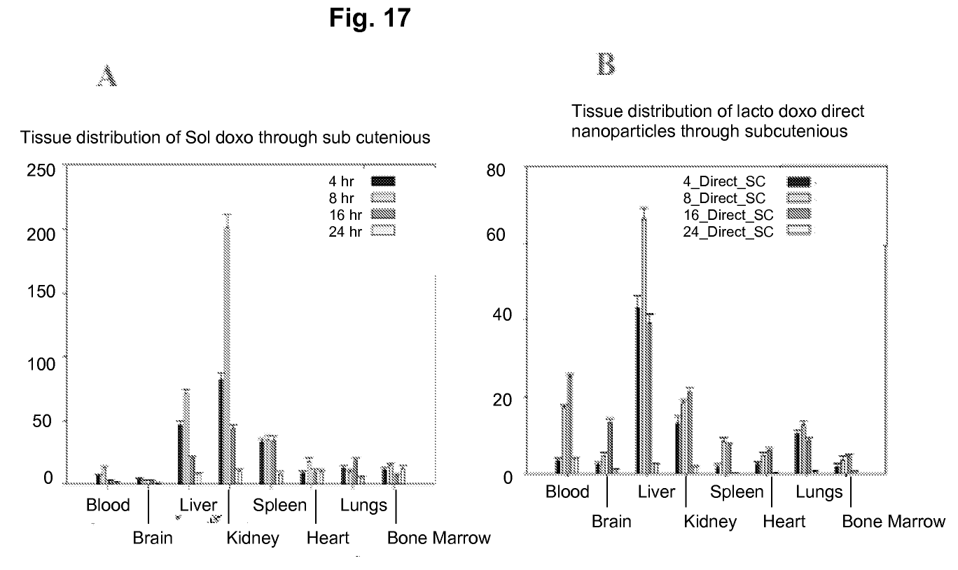

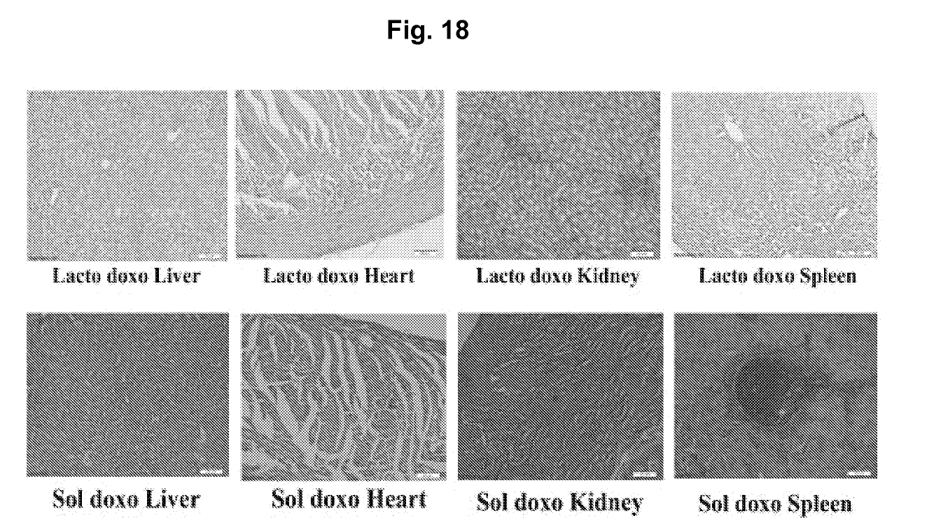

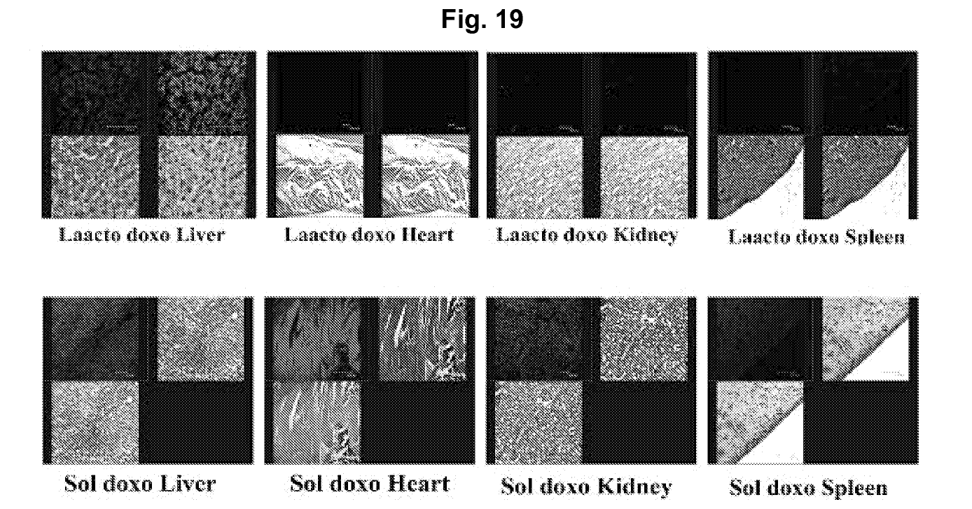

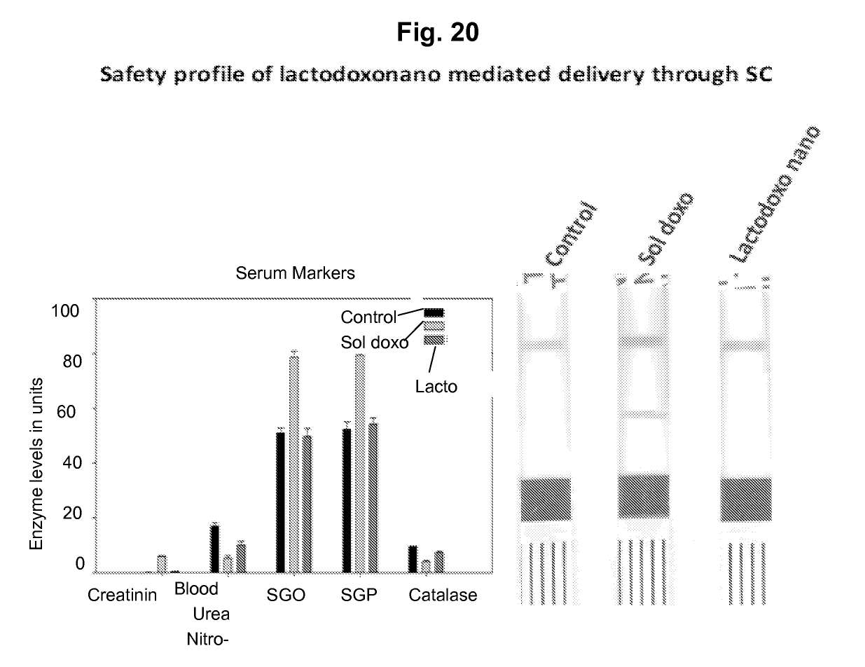

The nano particles of lactoferrin-doxorubicin were characterized as ˜68 nano meters. These nano particles are stable in water and get dissociated in acidic conditions (less than pH 3). Lacto-doxo nanoparticles are administered through sub cutenious and sacrifice the animal at indicated time points. Tissues like Brain, liver, heart, Kidney, Spleen, lungs, bone marrow and blood are collected, protein was precipitated in 30% AgNO₃ and drug was extracted in methanol. Drug was estimated with spectrophotometer show tissue distribution FIG. 17. Small piece of collected tissues are fixed in 4% PFA and embedded in paraffin stained with hematoxyline and eosin for slide preparation and gelatine coated slides. H & E coated slides observed under bright field microscope FIG. 18. These gelatine coated slides examined under Leica laser confocal microscope for drug and protein detection FIG. 19. Pharmacokinetics parameters shown in table-4. Safety analysis shows doxorubicin alone is cardiotoxic, nano form of doxo show no toxicity to heart FIG. 20.

TABLE 4

| | | | Lactodoxo | | LactoDoxo | | Lactodoxo |
|---|---|---|---|---|---|---|---|
| | Units | Sol doxo Blood | Blood | Sol doxo Liver | Liver | Sol doxo Heart | Heart |
| AUC | (h) * (μg/μL) | 167.94 | 19.12 | 1025 | 943.05 | 467.46 | 58.018 |
| AUMC | (h)\hat{ }2 * (μg/μL) | 1745.8 | 2007 | 10140 | 8772.1 | 13515 | 625.64 |
| Cmax | μg/μL | 12.855 | 10.915 | 71.123 | 73.373 | 18.198 | 5.0433 |
| Tmax | Hr | 8 | 8 | 8 | 8 | 8 | 8 |
| t½ | Hr | 7.0001 | 2.9224 | 5.9991 | 2.2411 | 18.662 | 5.0573 |
| MRT last | Hr | 10.394 | 10.393 | 9.8923 | 9.3018 | 28.912 | 10.784 |

| | | | LactoDoxo | | LactoDoxo |
|---|---|---|---|---|---|
| | Units | Sol doxo Kidney | Kidney | Sol doxo Spleen | Spleen |
| AUC | (h) * (μg/g) | 2249.1 | 76.291 | 553.3 | 29.329 |
| AUMC | (h)\hat{ }2 * (μg/g) | 19205 | 842.2 | 7927.3 | 242.22 |
| Cmax | Hr | 200.93 | 3.6453 | 34.747 | 2.1 |
| Tmax | μg/g | 8 | 8 | 8 | 8 |
| t½ | Hr | 3.888 | 3.5774 | 5.3088 | 1.8667 |
| MRT last | Hr | 8.5383 | 11.039 | 14.327 | 8.2587 |

Pharmacokinetics parameters
AUC: The integral of the concentration-time curve (after a single dose or in steady state).
T½: The time required for the concentration of the drug to reach half of its original value.
Cₘₐₓ: The peak plasma concentration of a drug after oral administration.
Tₘₐₓ: Time to reach Cₘₐₓ

### Example-8

### Oral Formulation Containing Doxorubicin Hydrochloride Loaded Lactoferrin Nano Particles for Treatment of Cancer

25 mg of lactoferrin in 100 μl of phosphate buffer saline was slowly mixed with the 5.3 mg of Doxorubicin hydrochloride in 100 μl of phosphate buffer saline and incubated in ice for 5 min. the mixture of lactoferrin and the drug was slowly added in steps of 0.01 ml to 15 ml of olive oil at 4° C. with continuous dispersion by gentle manual vortexing.

The particle formation of lactoferrin-Doxorubicin hydrochloride in oil phase was initiated by sonication using ranges from 50 to 80% power and the probe used is 0.375 inches diameter solid titanium tip Cat No. 0-120-0009 on Ultrasonic Homogenizer Model 300V/T of Bioloics Inc., USA and by passing the pulse for 2 second pulse and 2 seconds gap with continuously for 15 minutes. After sonication of the olive oil containing lactoferrin-Doxorubicin hydrochloride the resulting mixture was immediately frozen in liquid nitrogen at −196° C. for 10 min. Then it was transferred to ice and incubated for 4 hours. The Particles formed were pelleted by spinning at 5000 rpm for 10 minutes. The pellets of the composition of nano particles of lactoferrin and Doxorubicin hydrochloride obtained is decanted and was washed twice using 15 ml of ice cold diethyl ether. The pellets are immediately dispersed thoroughly by manual vortexing in 1 ml of phosphate buffered saline. The dispersed pellets were passed through the 0.2 micron filter and the filtrate obtained contained lactoferrin-Doxorubicin hydrochloride nano particles. These nano particles can be stored for one week at 4° C. and can be stored for more than a month at below 0° C. temperature.

The nano particles of lactoferrin-doxorubicin were characterized as ˜68 nano meters. These nano particles are stable in water and get dissociated in acidic conditions (less than pH 3). Lacto-doxo nanoparticles are administered liver cancer bearing wister rats through oral administration and sacrifice the animal after 10 doses. Tissues like Brain, liver, heart, Kidney, Spleen, lungs, bone marrow and blood are collected, protein was precipitated in 30% AgNO₃ and drug was extracted in methanol. Drug was estimated with spectrophotometer.

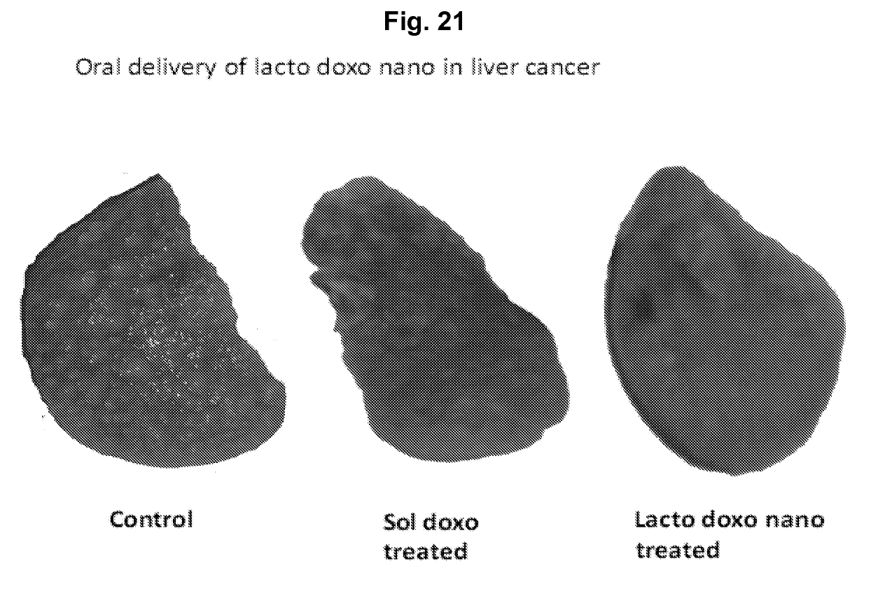

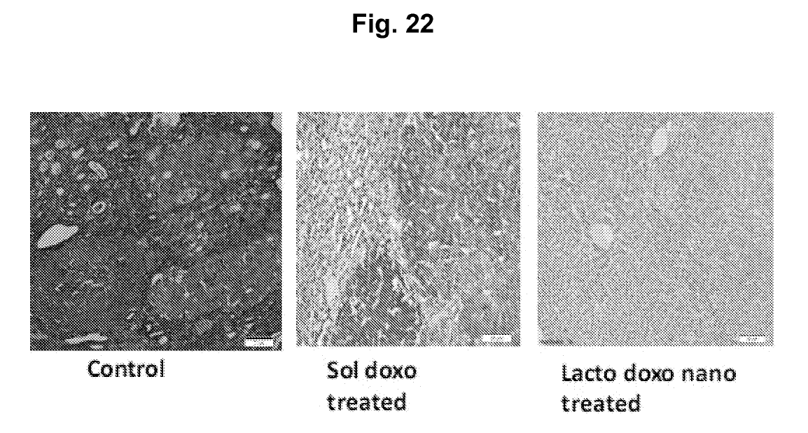

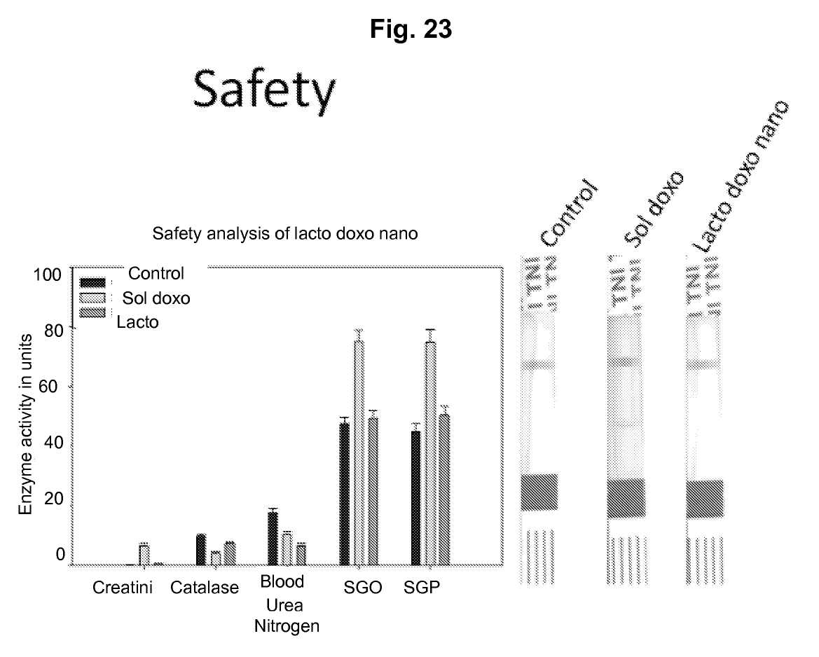

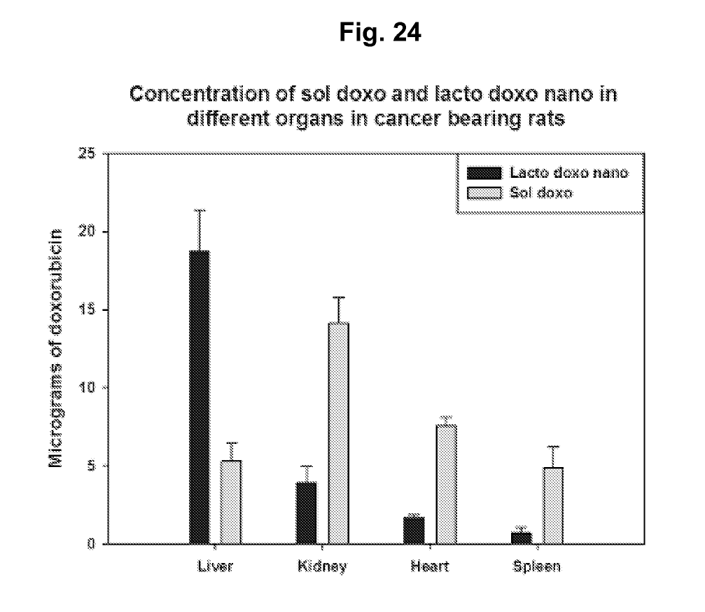

Small piece of collected tissues are fixed in 4% PFA and embedded in paraffin stained with hematoxyline and eosin for slide preparation. These slides examined under Leica laser confocal microscope for drug and protein detection. Cancer was confirmed by visualising the liver nodules shown in FIG. 21 by sacrificing liver cancer bearing rat. Tumour nodules present on the surface of liver and H&E stained slides of cancer liver shown in FIG. 22 observed under bright field microscope evaluated by pathologist from NIN, Hyderabad. Drug treatment was done every alternate day through oral. Safety analysis shows doxorubicin alone is cardiotoxic, nano form of doxo show no toxicity to heart FIG. 23. The regression was complete, rat did not show any reappearance of cancer out of 12 rats tested in contrast to the results of soluble lactoferrin mediated drug delivery as shown in FIG. 24.

### Example-9

### Intra Venous Formulation Containing Doxorubicin Hydrochloride Loaded Lactoferrin Nano Particles for Treatment of Cancer

25 mg of lactoferrin in 100 μl of phosphate buffer saline was slowly mixed with the 5.3 mg of Doxorubicin hydrochloride in 100 μl of phosphate buffer saline and incubated in ice for 5 min. the mixture of lactoferrin and the drug was slowly added in steps of 0.01 ml to 15 ml of olive oil at 4° C. with continuous dispersion by gentle manual vortexing.

The particle formation of lactoferrin-Doxorubicin hydrochloride in oil phase was initiated by sonication using ranges from 50 to 80% power and the probe used is 0.375 inches diameter solid titanium tip Cat No. 0-120-0009 on Ultrasonic Homogenizer Model 300V/T of Bioloics Inc., USA and by passing the pulse for 2 second pulse and 2 seconds gap with continuously for 15 minutes. After sonication of the olive oil containing lactoferrin-Doxorubicin hydrochloride the resulting mixture was immediately frozen in liquid nitrogen at −196° C. for 10 min. Then it was transferred to ice and incubated for 4 hours. The Particles formed were pelleted by spinning at 5000 rpm for 10 minutes. The pellets of the composition of nano particles of lactoferrin and Doxorubicin hydrochloride obtained is decanted and was washed twice using 15 ml of ice cold diethyl ether. The pellets are immediately dispersed thoroughly by manual vortexing in 1 ml of phosphate buffered saline. The dispersed pellets were passed through the 0.2 micron filter and the filtrate obtained contained lactoferrin-Doxorubicin hydrochloride nano particles. These nano particles can be stored for one week at 4° C. and can be stored for more than a month at below 0° C. temperature. These nanoparticles are lyophilized in the presence of mannitol. Lyophilized nanoparticles formulation can be dispersed freely in water or when administered orally. These nanoparticles are lyophilized in the presence of mannitol. Lyophilized nanoparticles formulation can be dispersed freely in water and administered intravenous route.

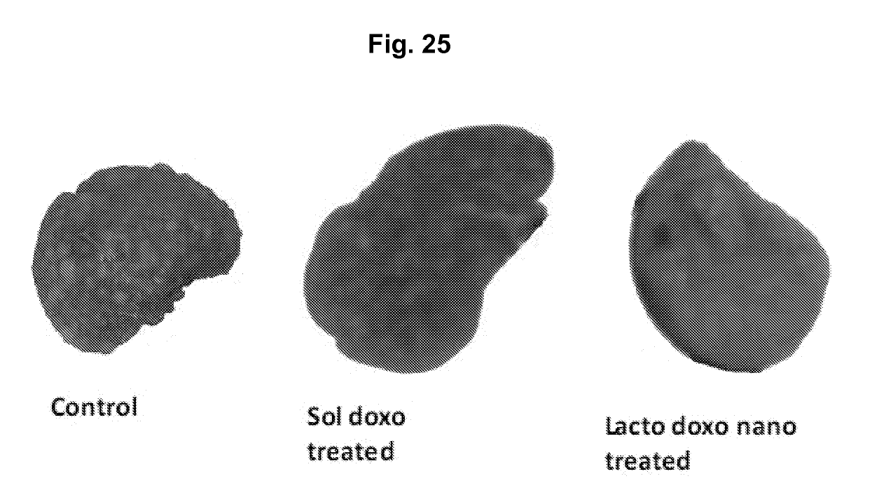

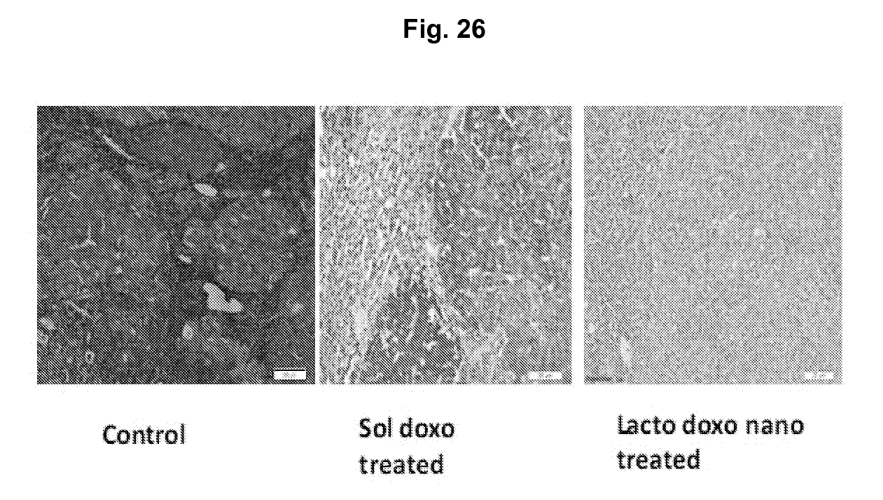

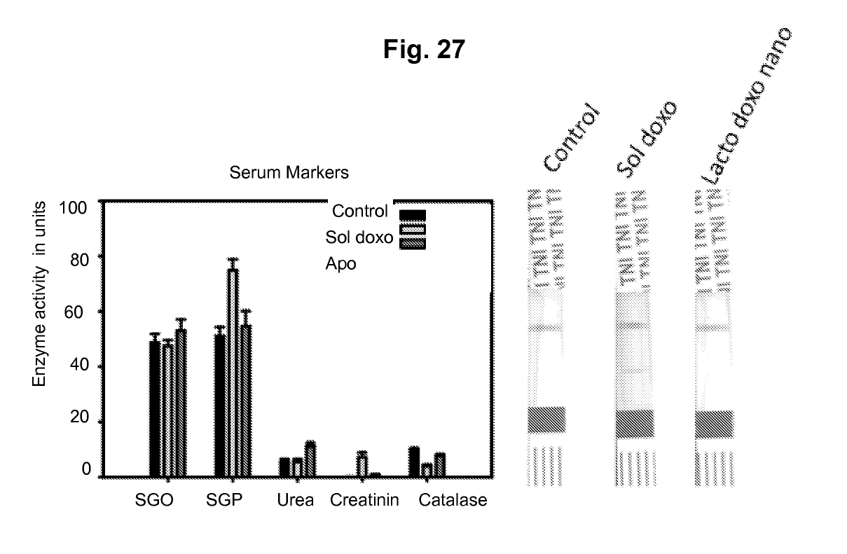

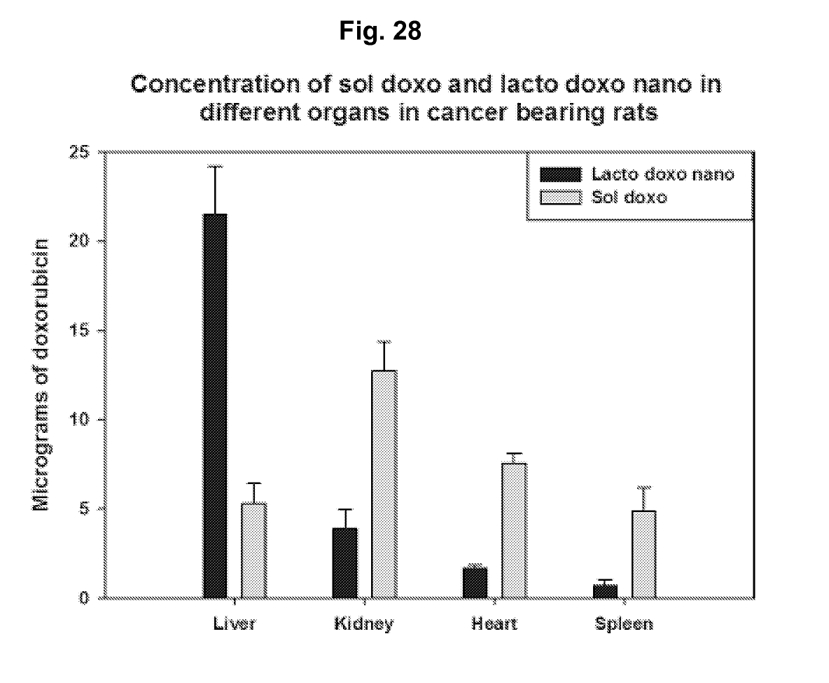

The nano particles of lactoferrin-doxorubicin were characterized as ˜68 nano meters. These nano particles are stable in water and get dissociated in acidic conditions (less than pH 3). Lacto-doxo nanoparticles are administered liver cancer bearing wister rats through intra venous administration and sacrifice the animal after 10 doses. Tissues like Brain, liver, heart, Kidney, Spleen, lungs, bone marrow and blood are collected, protein was precipitated in 30% AgNO₃ and drug was extracted in methanol. Drug was estimated with fluorimeter. Small piece of collected tissues are fixed in 4% PFA and embedded in paraffin stained with hematoxyline and eosin for slide preparation. These slides examined under Leica laser confocal microscope for drug and protein detection. Cancer was confirmed by visualising the liver nodules shown in FIG. 25 by sacrificing liver cancer bearing rat. Tumour nodules present on the surface of liver and H&E stained slides of cancer liver shown in FIG. 26 observed under bright field microscope evaluated by pathologist from NIN, Hyderabad. Drug treatment was done every alternate day through oral. Safety analysis shows doxorubicin alone is cardiotoxic, nano form of doxo show no toxicity to heart FIG. 27. The regression was complete, rat did not show any reappearance of cancer out of 12 rats tested in contrast to the results of soluble lactoferrin mediated drug delivery as shown in FIG. 28.

### Example 10

### Composition of Etoposide and Lactoferrin Nano Particles

25 mg of lactoferrin in 100 μl of phosphate buffer saline was slowly mixed with the 5.88 mg of ETOPOSIDE in 100 μl of DMSO and incubated in ice for 5 min. the mixture of lactoferrin and the drug was slowly added in steps of 0.01 ml to 15 ml of olive oil at 4° C. with continuous dispersion by gentle manual vortexing.

The particle of the lactoferrin-ETOPOSIDE in oil phase was initiated by sonication using ranges from 50 to 80% power and the probe used is 0.375 inches diameter solid titanium tip Cat No. 0-120-0009 on Ultrasonic Homogenizer Model 300V/T of Bioloics Inc., USA and by passing the pulse for 2 second pulse and 2 seconds gap with continuously for 15 minutes.

After sonication of the olive oil containing lactoferrin-ETOPOSIDE the resulting mixture was immediately frozen in liquid nitrogen at −196° C. for 10 min. Then it was transferred to ice and incubated for 4 hours. The Particles formed were pelleted by spinning at 6000 rpm for 10 minutes. The pellets of the composition of nano particles of lactoferrin and ETOPOSIDE obtained is decanted and was washed twice using 15 ml of ice cold diethyl ether. The pellets are immediately dispersed thoroughly by manual vortexing in 1 ml of phosphate buffered saline. The dispersed pellets were passed through the 0.2 micron filter and the filtrate obtained contained lactoferrin-ETOPOSIDE nano particles. These nano particles can be stored for one week at 4° C. and can be stored for more than a month at below 0° C. temperature.

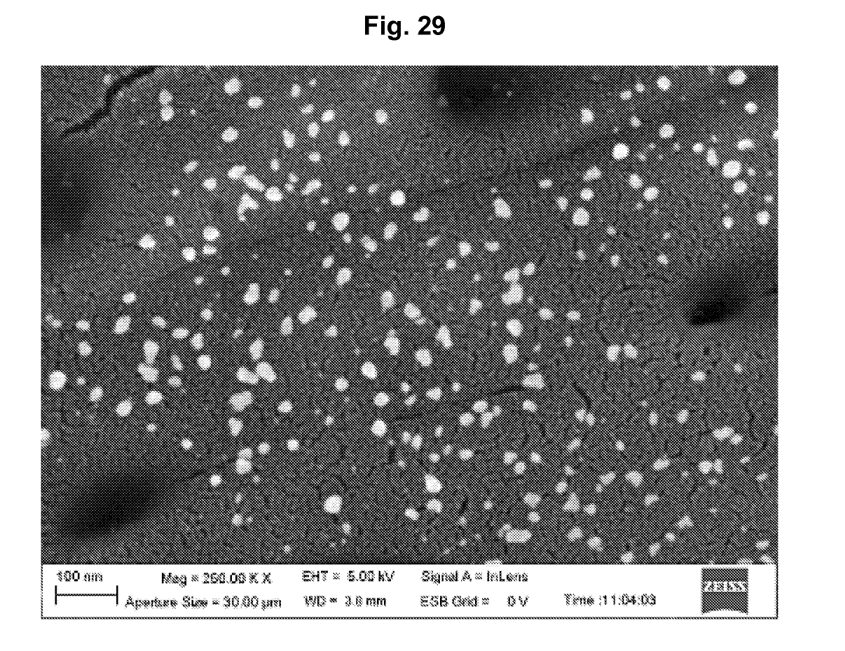

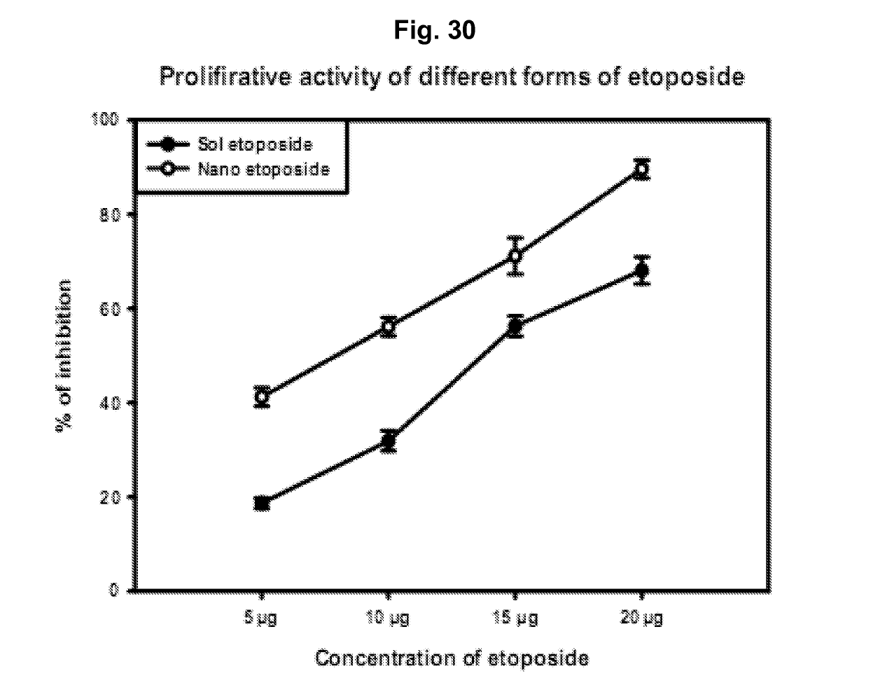

The nano particles of lactoferrin-ETOPOSIDE are characterized as ˜34 nano meters as shown in FIG. 29. These nano particles are stable in water and get dissociated in acidic conditions (less than pH 3). These particles inhibit proliferation of COLO 205 cell lines FIG. 30.

### Example 11

### Composition of Carboplatin and Lactoferrin Nano Particles

25 mg of lactoferrin in 100 μl of phosphate buffer saline was slowly mixed with the 3.71 mg of carboplatin in 100 μl of phosphate buffered saline and incubated in ice for 5 min. The mixture of lactoferrin and carboplatin was slowly added in steps of 0.01 ml to 15 ml of olive oil at 4° C. with continuous dispersion by gentle manual vortexing.

The particle of the lactoferrin-carboplatin in oil phase was initiated by sonication using ranges from 50 to 80% power and the probe used is 0.375 inches diameter solid titanium tip Cat No. 0-120-0009 on Ultrasonic Homogenizer Model 300V/T of Bioloics Inc., USA and by passing the pulse for 2 second pulse and 2 seconds gap with continuously for 15 minutes. After sonication of the olive oil containing lactoferrin-carboplatin, the resulting mixture was immediately frozen in liquid nitrogen at −196° C. for 10 min. Then it was transferred to ice and incubated for 4 hours. The particles formed were pelleted by spinning at 6000 rpm for 10 minutes. The pellets of the composition of nano particles of lactoferrin and carboplatin obtained is decanted and was washed twice using 15 ml of ice cold diethyl ether. The pellets are immediately dispersed thoroughly by manual vortexing in 1 ml of phosphate buffered saline.

The dispersed pellets were passed through the 0.2 micron filter and the filtrate obtained contained lactoferrin-carboplatin nano particles. These pellets can be stored for one week at 4° C. and can be stored for more than a month at below 0° C. temperature.

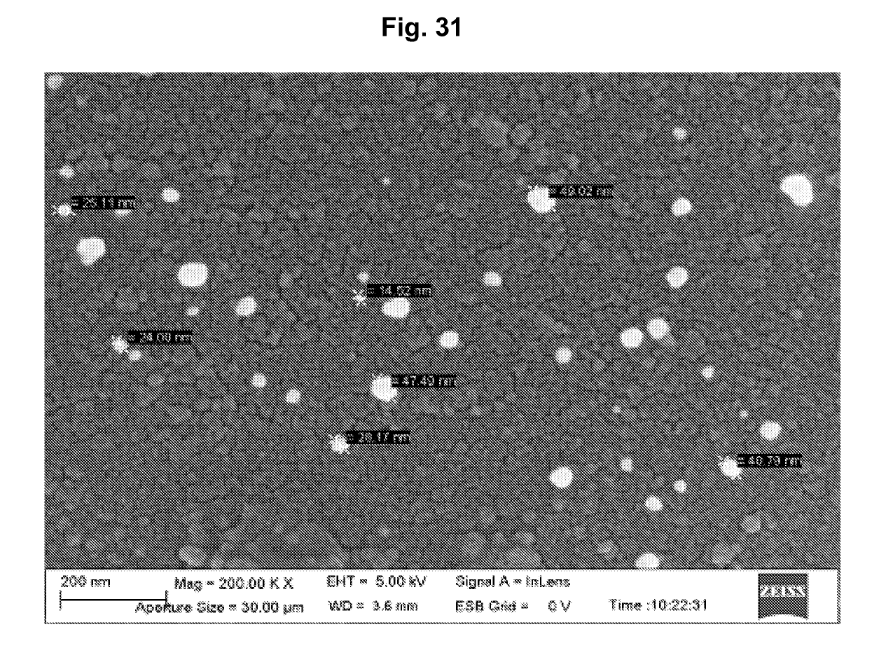

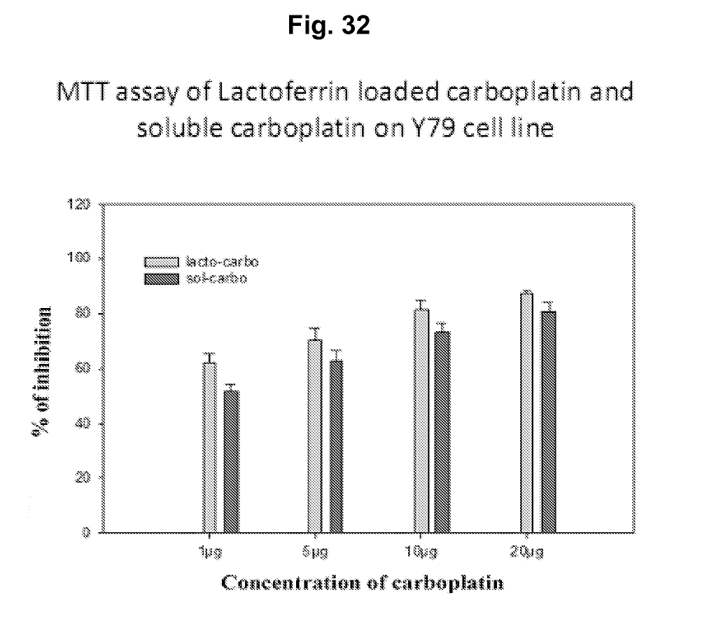

The nano particles of lactoferrin-Carboplatin are characterized as ˜60 nano meters as shown in FIG. 31. The pellets are stable in water and get dissociated in acidic conditions (less than pH 3). These particles inhibit proliferation of retino blastoma (Y79) cell lines as shown in FIG. 32. This Example also confirms that this method applicable to other platinum containing drugs like oxaliplatin and cisplatin also.

### Example-12

### Peri Occular Formulation Containing Doxorubicin Hydrochloride Loaded Lactoferrin Nano Particles for Localization in Eye

25 mg of lactoferrin in 100 μl of phosphate buffer saline was slowly mixed with the 5.3 mg of Doxorubicin hydrochloride in 100 μl of DMSO and incubated in ice for 5 min. the mixture of lactoferrin and the drug was slowly added in steps of 0.01 ml to 15 ml of olive oil at 4° C. with continuous dispersion by gentle manual vortexing.

The particle of the lactoferrin-Doxorubicin hydrochloride in oil phase was initiated by sonication using ranges from 50 to 80% power and the probe used is 0.375 inches diameter solid titanium tip Cat No. 0-120-0009 on Ultrasonic Homogenizer Model 300V/T of Bioloics Inc., USA and by passing the pulse for 2 second pulse and 2 seconds gap with continuously for 15 minutes. After sonication of the olive oil containing lactoferrin-Doxorubicin hydrochloride the resulting mixture was immediately frozen in liquid nitrogen at −196° C. for 10 min. Then it was transferred to ice and incubated for 4 hours. The Particles formed were pelleted by spinning at 5000 rpm for 10 minutes. The pellets of the composition of nano particles of lactoferrin and Doxorubicin hydrochloride obtained is decanted and was washed twice using 15 ml of ice cold diethyl ether. The pellets are immediately dispersed thoroughly by manual vortexing in 1 ml of phosphate buffered saline. The dispersed pellets were passed through the 0.2 micron filter and the filtrate obtained contained lactoferrin-Doxorubicin hydrochloride nano particles. These nano particles can be stored for one week at 4° C. and can be stored for more than a month at below 0° C. temperature.

These nanoparticles are lyophilized in the presence of mannitol. Lyophilized nanoparticles formulation can be dispersed freely in water and administered perioccular route.

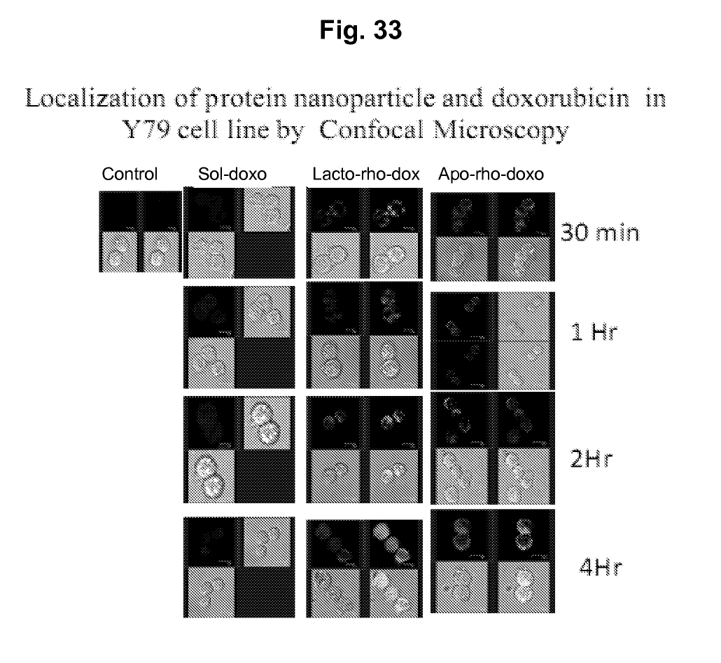

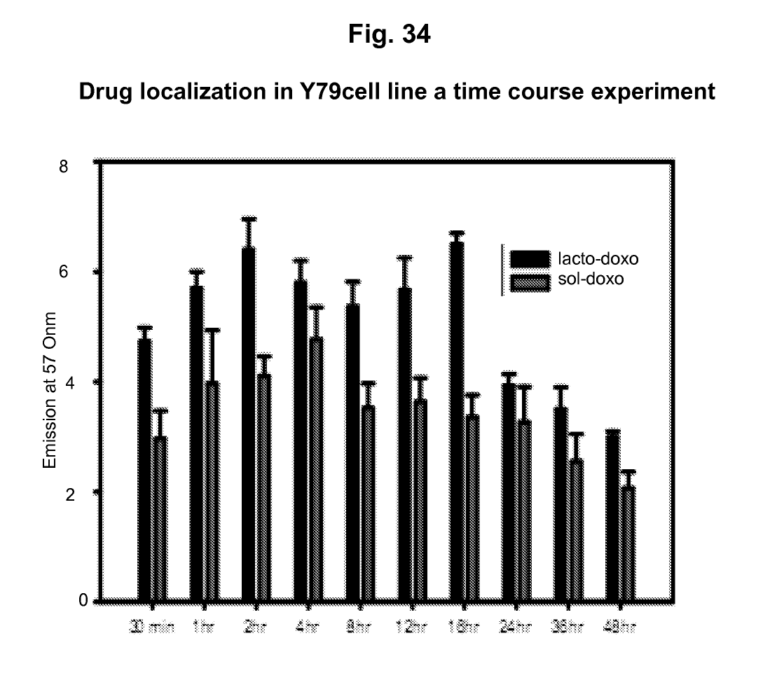

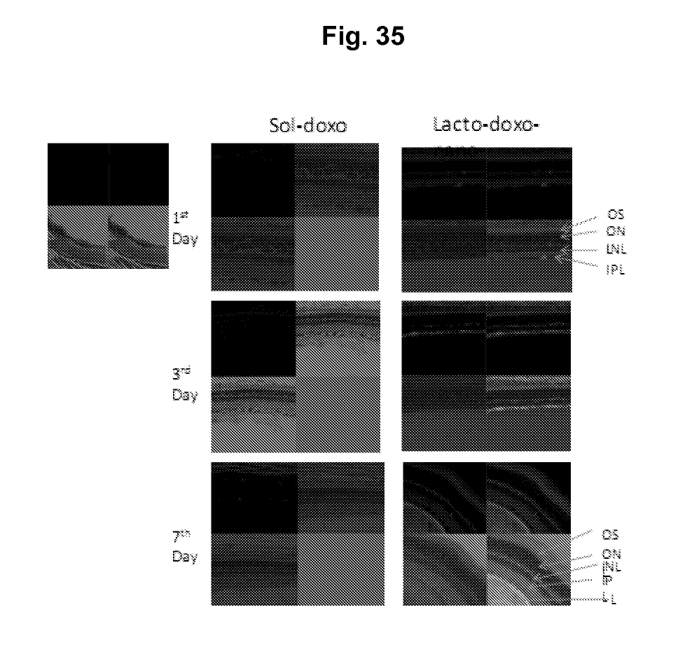

The nano particles of lactoferrin-Doxorubicin hydrochloride are characterized as ˜61 to 74 nano meters. These nano particles are stable in water and get dissociated in acidic conditions (less than pH 3). These particles show the cellular localization in retinoblastoma cell line (Y79) as shown in FIG. 33. These nano particles are stable in water and get dissociated in acidic conditions (less than pH 3). These particles shows good kinetics studies with of retino blastoma cell line as shown in FIG. 34. The nano particles of lactoferrin-Doxorubicin hydrochloride histological analysis show a strong association the rat reinal membrane (FIG. 35) suggesting the potential formulation in delivery drugs to eye.

### Example-13

### Composition of Paclitaxel and Lactoferrin Nano Particles

25 mg of lactoferrin in 100 μl of phosphate buffer saline was slowly mixed with the 10 mg of PACLITAXEL in 100 μl of DMSO and incubated in ice for 5 min. the mixture of lactoferrin and the drug was slowly added in steps of 0.01 ml to 15 ml of olive oil at 4° C. with continuous dispersion by gentle manual vortexing.

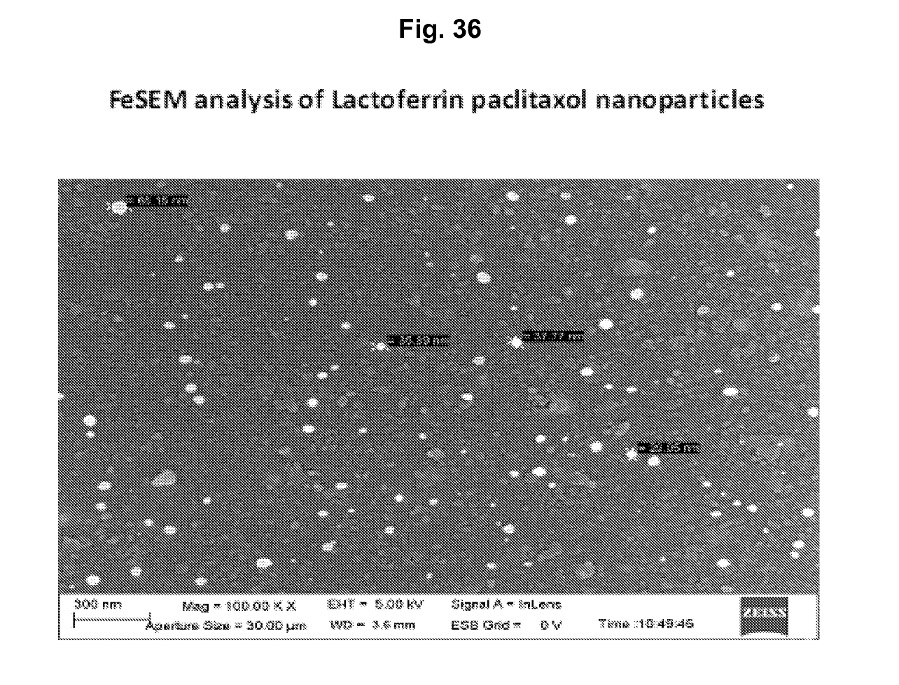

The particle of the lactoferrin-PACLITAXEL in oil phase was initiated by sonication using ranges from 50 to 80% power and the probe used is 0.375 inches diameter solid titanium tip Cat No. 0-120-0009 on Ultrasonic Homogenizer Model 300V/T of Bioloics Inc., USA and by passing the pulse for 2 second pulse and 2 seconds gap with continuously for 15 minutes. After sonication of the olive oil containing lactoferrin-PACLITAXEL the resulting mixture was immediately frozen in liquid nitrogen at −196° C. for 10 min. Then it was transferred to ice and incubated for 4 hours. The Particles formed were pelleted by spinning at 6000 rpm for 10 minutes. The pellets of the composition of nano particles of lactoferrin and PACLITAXEL obtained is decanted and was washed twice using 15 ml of ice cold diethyl ether. The pellets are immediately dispersed thoroughly by manual vortexing in 1 ml of phosphate buffered saline. The dispersed pellets were passed through the 0.2 micron filter and the filtrate obtained contained lactoferrin-PACLITAXEL nano particles. These nano particles can be stored for one week at 4° C. and can be stored for more than a month at below 0° C. temperature. The nano particles of lactoferrin-PACLITAXEL are characterized as 74 to 82 nano meters as shown in FIG. 36.

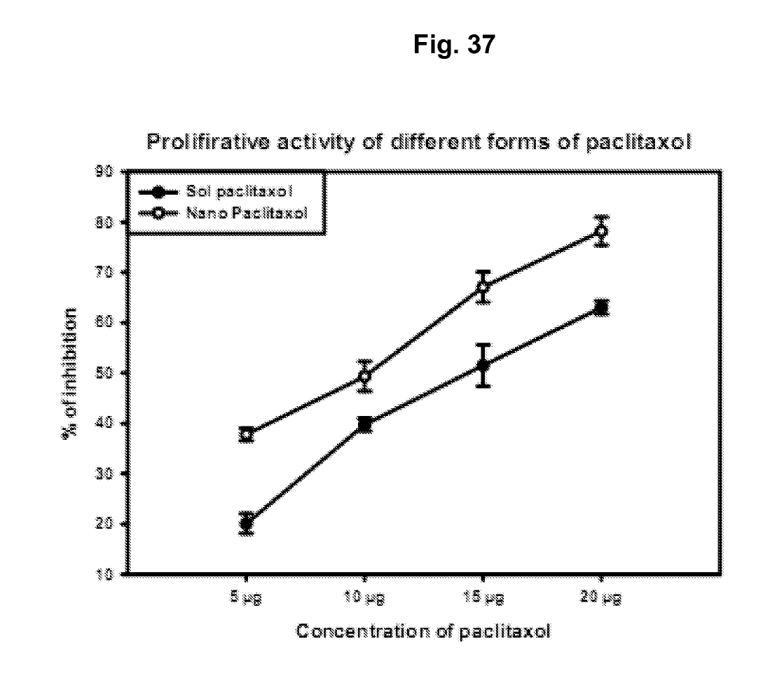

These nano particles are stable in water and get dissociated in acidic conditions (less than pH 3). These particles inhibit proliferation of SupT1 T cell lymphoma cell line as shown in FIG. 37.

### Example-14

### Composition of 5-Fluoro Uracil and Lactoferrin Nano Particles

25 mg of lactoferrin in 100 μl of phosphate buffer saline was slowly mixed with the 10 mg of 5-FLUORO URACIL in 100 μl of DMSO and incubated in ice for 5 min. the mixture of lactoferrin and the drug was slowly added in steps of 0.01 ml to 15 ml of olive oil at 4° C. with continuous dispersion by gentle manual vortexing.

The particle of the lactoferrin-5-FLUORO URACIL in oil phase was initiated by sonication using ranges from 50 to 80% power and the probe used is 0.375 inches diameter solid titanium tip Cat No. 0-120-0009 on Ultrasonic Homogenizer Model 300V/T of Bioloics Inc., USA and by passing the pulse for 2 second pulse and 2 seconds gap with continuously for 15 minutes. After sonication of the olive oil containing lactoferrin-5-FLUORO URACIL the resulting mixture was immediately frozen in liquid nitrogen at −196° C. for 10 min. Then it was transferred to ice and incubated for 4 hours. The Particles formed were pelleted by spinning at 5000 rpm for 10 minutes. The pellets of the composition of nano particles of lactoferrin and 5-FLUORO URACIL obtained is decanted and was washed twice using 15 ml of ice cold diethyl ether. The pellets are immediately dispersed thoroughly by manual vortexing in 1 ml of phosphate buffered saline. The dispersed pellets were passed through the 0.2 micron filter and the filtrate obtained contained lactoferrin-5-FLUORO URACIL nano particles. These nano particles can be stored for one week at 4° C. and can be stored for more than a month at below 0° C. temperature.

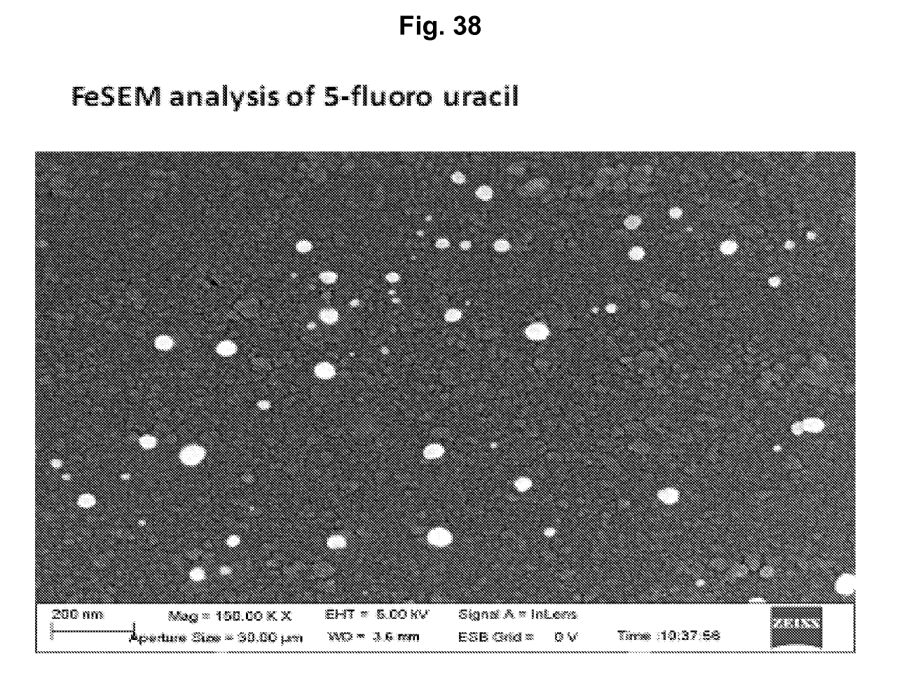

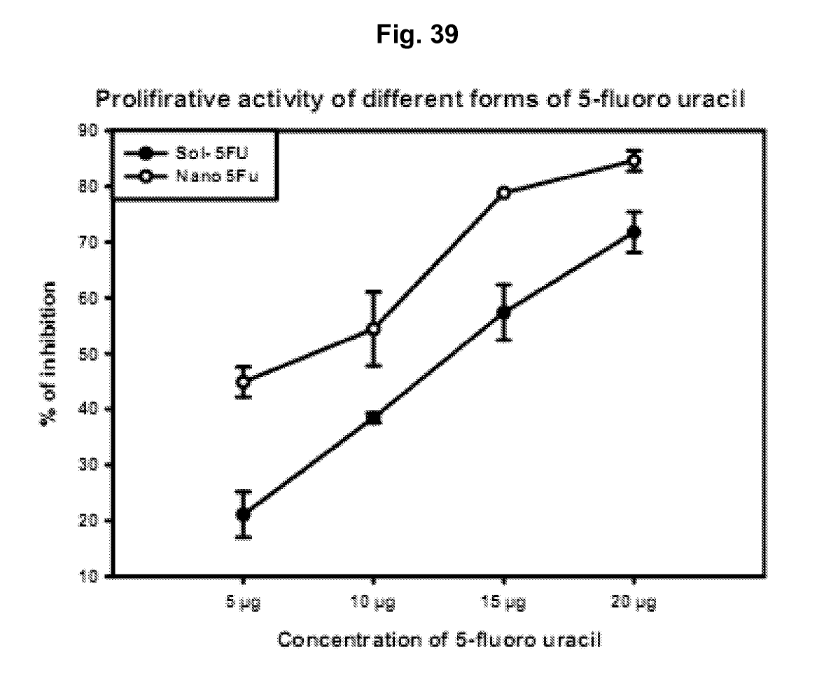

The nano particles of lactoferrin-5-FLUORO URACIL are characterized as 74 to 82 nano meters as shown in FIG. 38. These nano particles are stable in water and get dissociated in acidic conditions (less than pH 3). These particles inhibit proliferation of SupT1 T cell lymphoma cell line as shown in FIG. 39.

### Example-15

### Composition of Cyclophosphomide and Lactoferrin Nano Particles

25 mg of lactoferrin in 100 μl of phosphate buffer saline was slowly mixed with the 10 mg of CYCLOPHOSPHOMIDE in 100 μl of 1×PBS and incubated in ice for 5 min. the mixture of lactoferrin and the drug was slowly added in steps of 0.01 ml to 15 ml of olive oil at 4° C. with continuous dispersion by gentle manual vortexing.

The particle of the lactoferrin-CYCLOPHOSPHOMIDE in oil phase was initiated by sonication using ranges from 50 to 80% power and the probe used is 0.375 inches diameter solid titanium tip Cat No. 0-120-0009 on Ultrasonic Homogenizer Model 300V/T of Bioloics Inc., USA and by passing the pulse for 2 second pulse and 2 seconds gap with continuously for 15 minutes. After sonication of the olive oil containing lactoferrin-CYCLOPHOSPHOMIDE the resulting mixture was immediately frozen in liquid nitrogen at −196° C. for 10 min. Then it was transferred to ice and incubated for 4 hours. The Particles formed were pelleted by spinning at 6000 rpm for 10 minutes. The pellets of the composition of nano particles of lactoferrin and CYCLOPHOSPHOMIDE obtained is decanted and was washed twice using 15 ml of ice cold diethyl ether. The pellets are immediately dispersed thoroughly by manual vortexing in 1 ml of phosphate buffered saline. The dispersed pellets were passed through the 0.2 micron filter and the filtrate obtained contained lactoferrin-CYCLOPHOSPHOMIDE nano particles. These nano particles can be stored for one week at 4° C. and can be stored for more than a month at below 0° C. temperature.

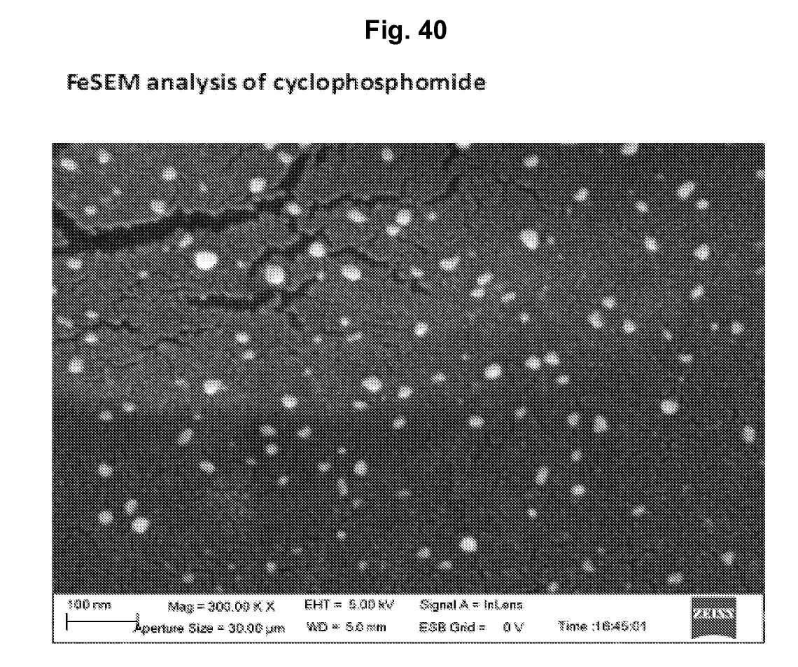

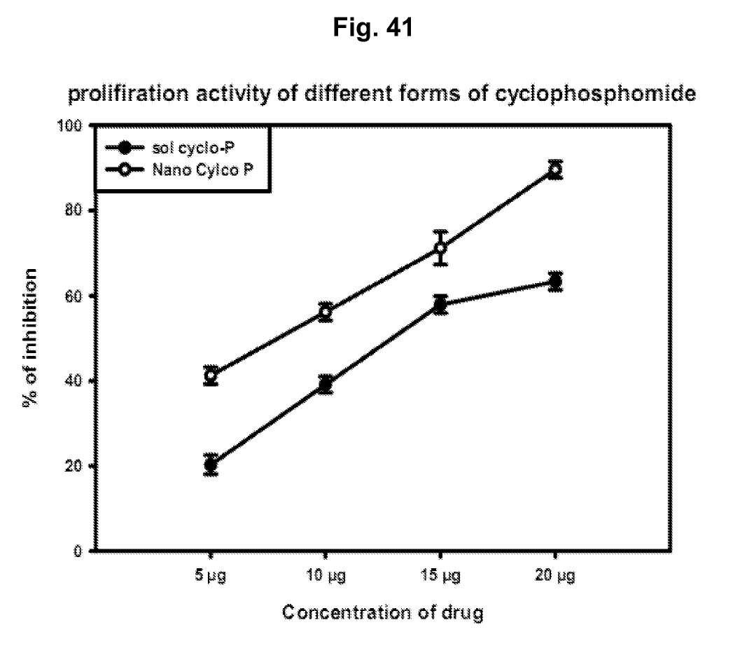

The nano particles of lactoferrin-CYCLOPHOSPHOMIDE are characterized as 74 to 82 nano meters as shown in FIG. 40. These nano particles are stable in water and get dissociated in acidic conditions (less than pH 3). These particles inhibit proliferation of colo 205 cell line as shown in FIG. 41.

### Example-16

### Composition of Gemcitabine and Lactoferrin Nano Particles

25 mg of lactoferrin in 100 μl of phosphate buffer saline was slowly mixed with the 10 mg of GEMCITABINE in 100 μl of DMSO and incubated in ice for 5 min. the mixture of lactoferrin and the drug was slowly added in steps of 0.01 ml to 15 ml of olive oil at 4° C. with continuous dispersion by gentle manual vortexing.

The particle of the lactoferrin-GEMCITABINE in oil phase was initiated by sonication using ranges from 50 to 80% power and the probe used is 0.375 inches diameter solid titanium tip Cat No. 0-120-0009 on Ultrasonic Homogenizer Model 300V/T of Bioloics Inc., USA and by passing the pulse for 2 second pulse and 2 seconds gap with continuously for 15 minutes. After sonication of the olive oil containing lactoferrin-GEMCITABINE the resulting mixture was immediately frozen in liquid nitrogen at −196° C. for 10 min. Then it was transferred to ice and incubated for 4 hours. The Particles formed were pelleted by spinning at 5000 rpm for 10 minutes. The pellets of the composition of nano particles of lactoferrin and GEMCITABINE obtained is decanted and was washed twice using 15 ml of ice cold diethyl ether. The pellets are immediately dispersed thoroughly by manual vortexing in 1 ml of phosphate buffered saline. The dispersed pellets were passed through the 0.2 micron filter and the filtrate obtained contained lactoferrin-GEMCITABINE nano particles. These nano particles can be stored for one week at 4° C. and can be stored for more than a month at below 0° C. temperature.

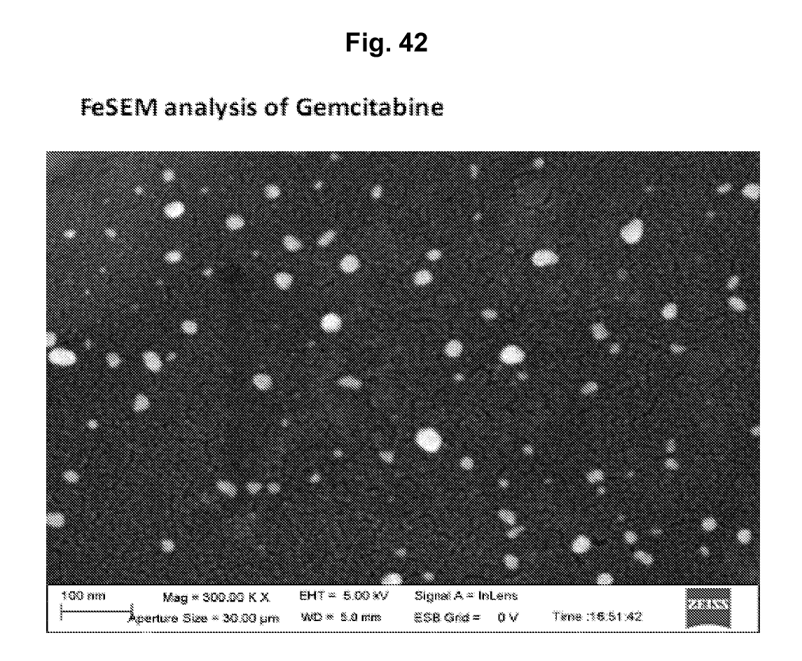

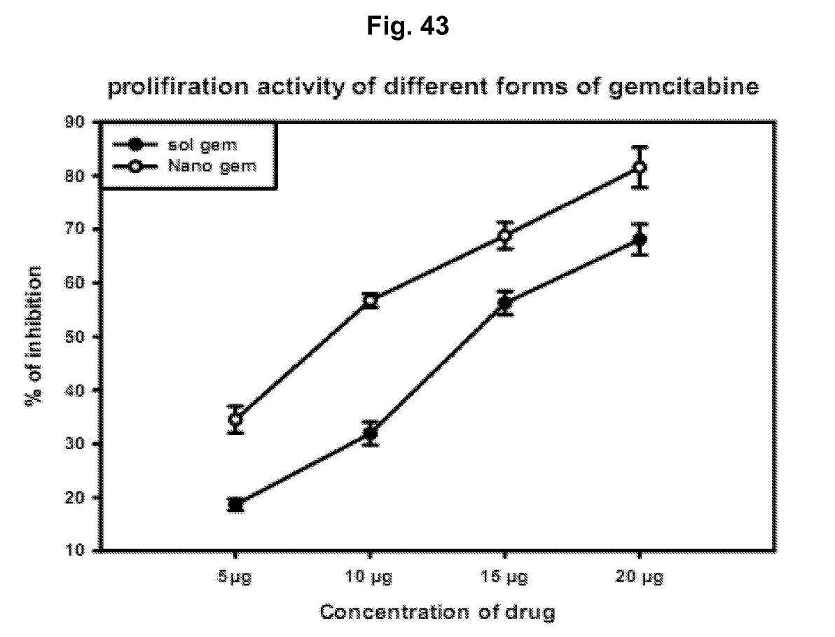

The nano particles of lactoferrin-GEMCITABINE are characterized as 74 to 82 nano meters as shown in FIG. 42. These nano particles are stable in water and get dissociated in acidic conditions (less than pH 3). These particles inhibit proliferation of COLO 205 cell line as shown in FIG. 43.

### Example-17

### Composition of Cisplatin and Lactoferrin Nano Particles

25 mg of lactoferrin in 100 μl of phosphate buffer saline was slowly mixed with the 10 mg of CISPLATIN in 100 μl of DMSO and incubated in ice for 5 min. the mixture of lactoferrin and the drug was slowly added in steps of 0.01 ml to 15 ml of olive oil at 4° C. with continuous dispersion by gentle manual vortexing.

The particle of the lactoferrin-CISPLATIN in oil phase was initiated by sonication using ranges from 50 to 80% power and the probe used is 0.375 inches diameter solid titanium tip Cat No. 0-120-0009 on Ultrasonic Homogenizer Model 300V/T of Bioloics Inc., USA and by passing the pulse for 2 second pulse and 2 seconds gap with continuously for 15 minutes. After sonication of the olive oil containing lactoferrin-CISPLATIN the resulting mixture was immediately frozen in liquid nitrogen at −196° C. for 10 min. Then it was transferred to ice and incubated for 4 hours. The Particles formed were pelleted by spinning at 5000 rpm for 10 minutes. The pellets of the composition of nano particles of lactoferrin and CISPLATIN obtained is decanted and was washed twice using 15 ml of ice cold diethyl ether. The pellets are immediately dispersed thoroughly by manual vortexing in 1 ml of phosphate buffered saline. The dispersed pellets were passed through the 0.2 micron filter and the filtrate obtained contained lactoferrin-CISPLATIN nano particles. These nano particles can be stored for one week at 4° C. and can be stored for more than a month at below 0° C. temperature.

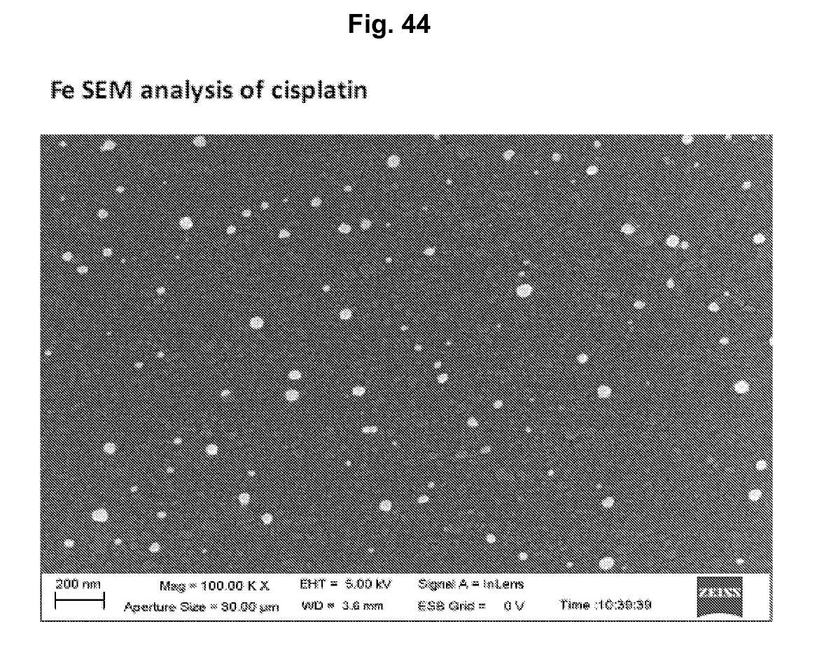

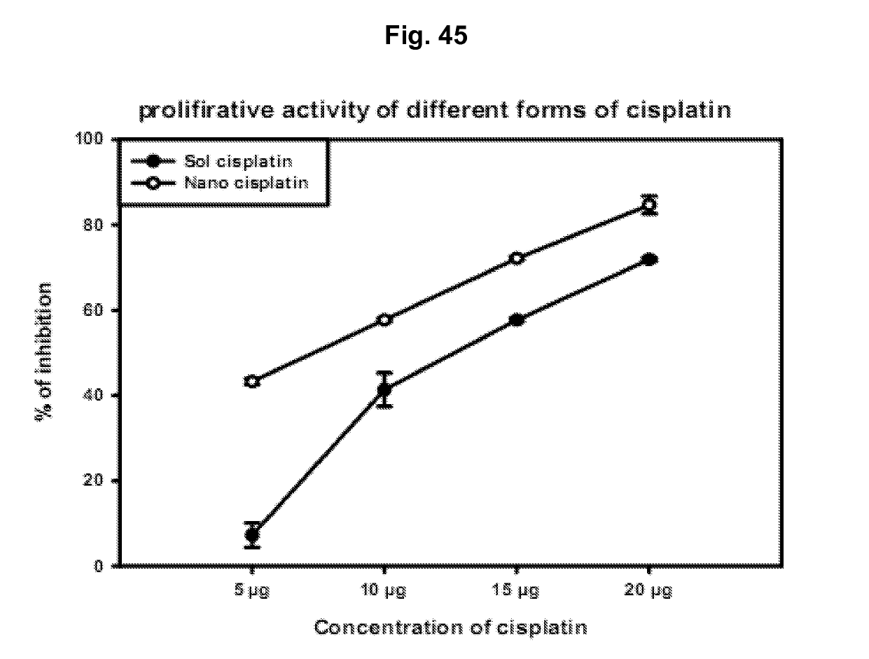

The nano particles of lactoferrin-CISPLATIN are characterized as 74 to 82 nano meters as shown in FIG. 44. These nano particles are stable in water and get dissociated in acidic conditions (less than pH 3). These particles inhibit proliferation of COLO 205 cell line as shown in FIG. 45.

### Example-18

### Composition of Methotraxate and Lactoferrin Nano Particles

25 mg of lactoferrin in 100 μl of phosphate buffer saline was slowly mixed with the 10 mg of METHOTRAXATE in 100 μl of DMSO and incubated in ice for 5 min. the mixture of lactoferrin and the drug was slowly added in steps of 0.01 ml to 15 ml of olive oil at 4° C. with continuous dispersion by gentle manual vortexing.

The particle of the lactoferrin-METHOTRAXATE in oil phase was initiated by sonication using ranges from 50 to 80% power and the probe used is 0.375 inches diameter solid titanium tip Cat No. 0-120-0009 on Ultrasonic Homogenizer Model 300V/T of Bioloics Inc., USA and by passing the pulse for 2 second pulse and 2 seconds gap with continuously for 15 minutes. After sonication of the olive oil containing lactoferrin-METHOTRAXATE the resulting mixture was immediately frozen in liquid nitrogen at −196° C. for 10 min. Then it was transferred to ice and incubated for 4 hours. The Particles formed were pelleted by spinning at 5000 rpm for 10 minutes. The pellets of the composition of nano particles of lactoferrin and METHOTRAXATE obtained is decanted and was washed twice using 15 ml of ice cold diethyl ether. The pellets are immediately dispersed thoroughly by manual vortexing in 1 ml of phosphate buffered saline. The dispersed pellets were passed through the 0.2 micron filter and the filtrate obtained contained lactoferrin-METHOTRAXATE nano particles. These nano particles can be stored for one week at 4° C. and can be stored for more than a month at below 0° C. temperature.

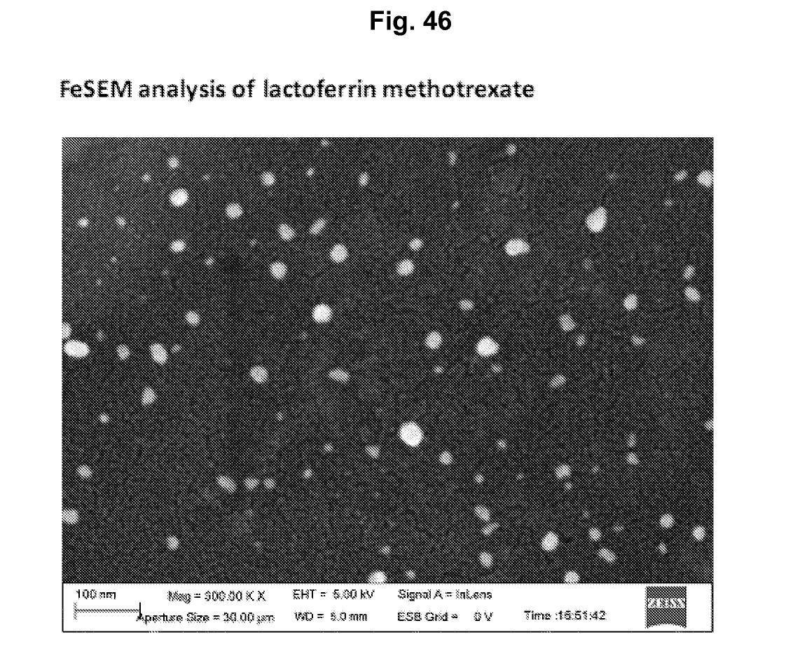

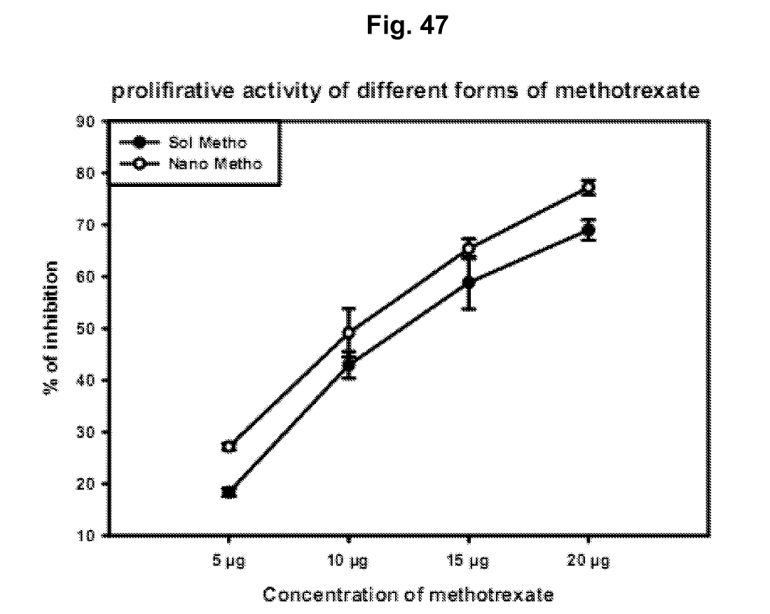

The nano particles of lactoferrin-METHOTRAXATE are characterized as 74 to 82 nano meters as shown in FIG. 46. These nano particles are stable in water and get dissociated in acidic conditions (less than pH 3). These particles inhibit proliferation of SupT1 T cell lymphoma cell line as shown in FIG. 47.

### Example-19

### Composition of Cytarabine and Lactoferrin Nano Particles

25 mg of lactoferrin in 100 μl of phosphate buffer saline was slowly mixed with the 10 mg of cytarabine in 100 μl of DMSO and incubated in ice for 5 min. the mixture of lactoferrin and the drug was slowly added in steps of 0.01 ml to 15 ml of olive oil at 4° C. with continuous dispersion by gentle manual vortexing.

The particle of the lactoferrin-cytarabine in oil phase was initiated by sonication using ranges from 50 to 80% power and the probe used is 0.375 inches diameter solid titanium tip Cat No. 0-120-0009 on Ultrasonic Homogenizer Model 300V/T of Bioloics Inc., USA and by passing the pulse for 2 second pulse and 2 seconds gap with continuously for 15 minutes. After sonication of the olive oil containing lactoferrin-cytarabine the resulting mixture was immediately frozen in liquid nitrogen at −196° C. for 10 min. Then it was transferred to ice and incubated for 4 hours. The Particles formed were pelleted by spinning at 5000 rpm for 10 minutes. The pellets of the composition of nano particles of lactoferrin and cytarabine obtained is decanted and was washed twice using 15 ml of ice cold diethyl ether. The pellets are immediately dispersed thoroughly by manual vortexing in 1 ml of phosphate buffered saline. The dispersed pellets were passed through the 0.2 micron filter and the filtrate obtained contained lactoferrin-cytarabine nano particles. These nano particles can be stored for one week at 4° C. and can be stored for more than a month at below 0° C. temperature.

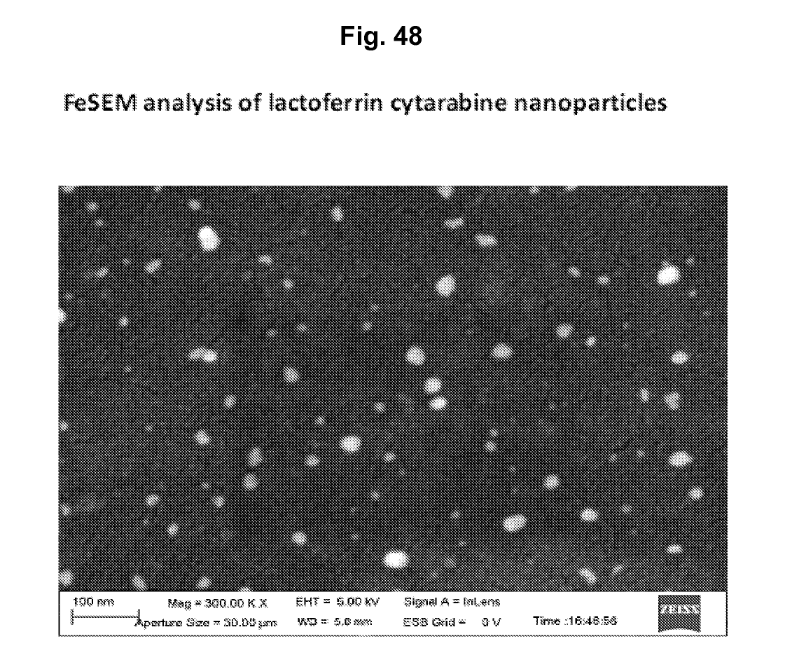

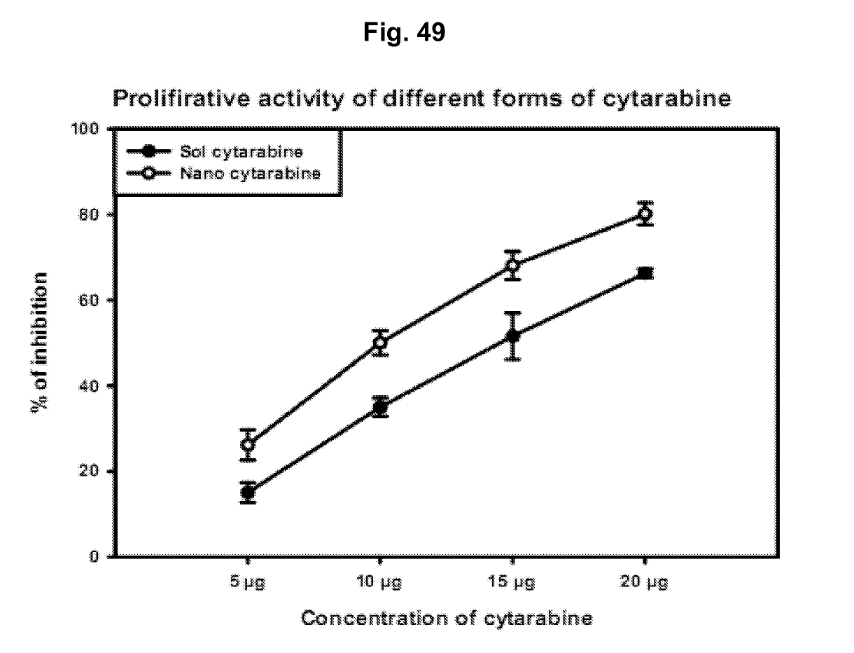

The nano particles of lactoferrin-cytarabine are characterized as 74 to 82 nano meters as shown in FIG. 48. These nano particles are stable in water and get dissociated in acidic conditions (less than pH 3). These particles inhibit proliferation of SupT1 T cell lymphoma cell line as shown in FIG. 49.

### Example-20

### Composition of Anti Hiv-1 Drugs and Peptides with Lactoferrin Nano Particles

25 mg of lactoferrin in 100 μl of phosphate buffer saline was slowly mixed with the 5 μM of AZT and 250 ng of T20 incubated in ice for 5 min. the mixture of lactoferrin and the drug was slowly added in steps of 0.01 ml to 15 ml of olive oil at 4° C. with continuous dispersion by gentle manual vortexing.

The particle of the lactoferrin-HIV drugs and peptides in oil phase was initiated by sonication using ranges from 50 to 80% power and the probe used is 0.375 inches diameter solid titanium tip Cat No. 0-120-0009 on Ultrasonic Homogenizer Model 300V/T of Bioloics Inc., USA and by passing the pulse for 2 second pulse and 2 seconds gap with continuously for 15 minutes. After sonication of the olive oil containing lactoferrin-HIV drugs and peptides the resulting mixture was immediately frozen in liquid nitrogen at −196° C. for 10 min. Then it was transferred to ice and incubated for 4 hours. The Particles formed were pelleted by spinning at 5000 rpm for 10 minutes. The pellets of the composition of nano particles of lactoferrin and HIV drugs and peptides obtained is decanted and was washed twice using 15 ml of ice cold diethyl ether. The pellets are immediately dispersed thoroughly by manual vortexing in 1 ml of phosphate buffered saline. The dispersed pellets were passed through the 0.2 micron filter and the filtrate obtained contained lactoferrin-HIV drugs and peptides nano particles. These nano particles can be stored for one week at 4° C. and can be stored for more than a month at below 0° C. temperature.

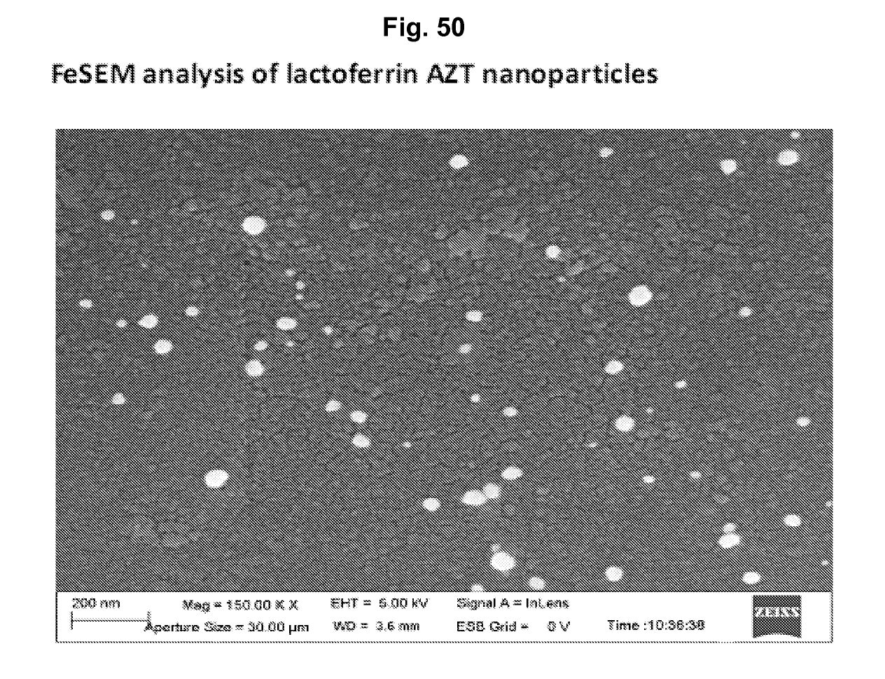

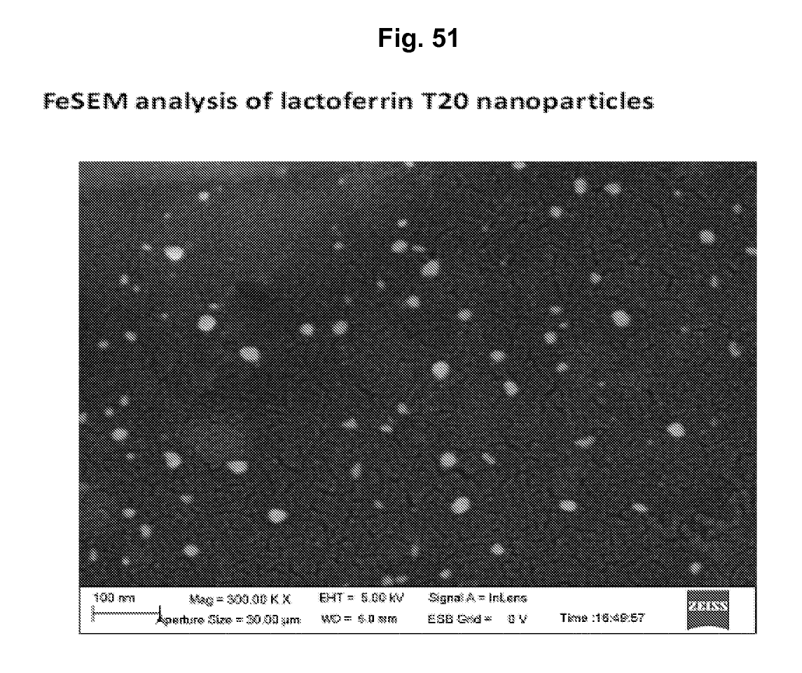

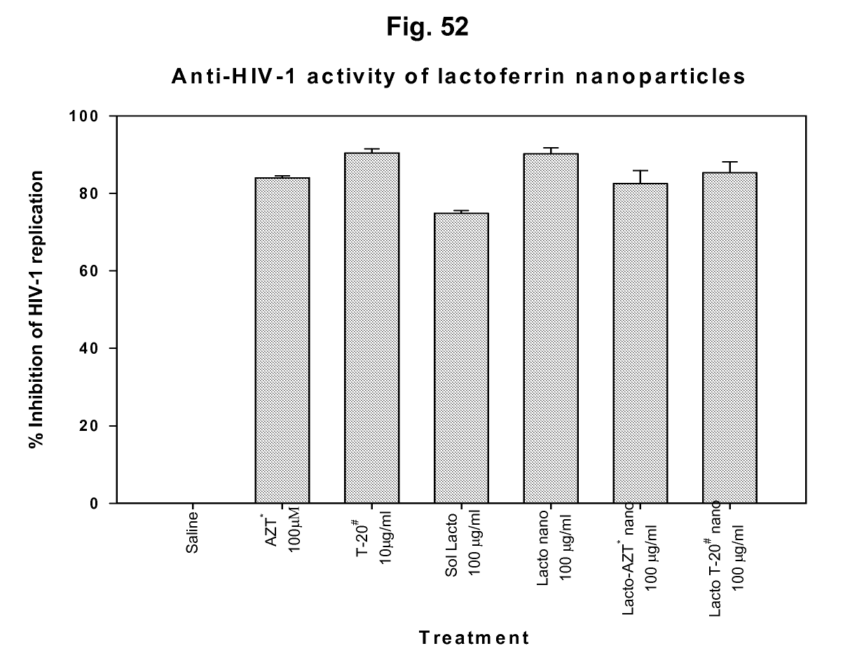

The nano particles of lactoferrin nanoparticles characterized as 74 to 82 nano meters. Lctoferrin-AZTand lactoferrin T₂₀ are characterized as 74 to 82 nano meters as shown in FIG. 50-51. These nano particles are stable in water and get dissociated in acidic conditions (less than pH 3). These particles inhibit HIV-1 activity as shown in FIG. 52.

### Experimental Details

For studying the efficacy of the composition of the present invention described in the above said Examples, the following experiments were conducted
A) Nanoparticles of in lactoferrin and drug referred in Examples 3-20 and lactoferrin alone referred to Example 1 and 2 were characterized for their size with Scanning Electron Microscopy (SEM), Atomic force microscopy and Trasnmission electron microscopy (TEM).
B) The Characterization of nanoparticles of lactoferrin without and with drug referred in Examples 1 and 3, for the presence of Lactoferrin was done by dot blot assay using Mouse anti-human lactoferrin monoclonal antibody.
C) The amount of the drug used and protein in nano particles of lactoferrin and drug referred in Examples 3-13 and lactoferrin alone referred in Examples 1 and 2 was quantified for absorption spectra of protein and drug using drug spectrum.
D) Particle localization in cells was done by incubating 1 million cells such as SupT1 (Non Hodgkins T cell lymphoma cell line) and other cell lines with drug loaded particles referred in Example 4 and morphology is recorded at various timepoints. The control cells, which are untreated cells, are also recorded at the various time points.
   1 million cells (SupT1) are incubated with drug-loaded particles and taken out at various time points as indicated in. Spinning at 2000 rpm collects the cells and the cells are washed thrice with TBS and the cells are observed for fluorescence under laser confocal microscope. These results suggest the auto fluorescence of drug (due to organic moiety) is localized in cytosol of the cell in a time dependent manner.
E) Effect of nanoparticles in drug delivery referred to Examples 3, 10 to 19 was observed by seeding cancer cells. The results showed that the drug in nanoparticle form efficiently inhibit the growth of SupT1cells/neuroblastoma/ratinoblstoma cancer cells compared to drug alone.
F) Localization of nanoparticles and drug in cancer tissues as referred in examples 4 to 7 (healthy rats), 8 and 9 (cancer rats) was carried out by administration of formulation to rat as per route indicated at single dose followed by histochemical analysis of tissues, and extraction of drug from the tissue and estimating drug concentrations at indicted time points followed by pharmacokinetic analysis using Kinetica v5.0 software.
G) Experiment to show regression of hepatoma in rat by drug loaded

Lactoferrin particles referred in example 8 and 9.

Rat Hepatocellular Carcinoma Model Generation—

Hepatocellular carcinoma was induced by 100 mg/L of diethylnitrosamine in drinking water for 8-weeks to the 2 months old Wistar rat.

Testing the Efficacy of the Composition Referred in Example 8 and 9 During the Cancer Progression

Hepatocellular carcinoma was induced by 100 mg/L of diethylnitrosamine in drinking water for 8-weeks to the 2 month's old Wister rat.

The experiment was repeated in the group of six/five animals and the results are statistically significant proving that nano particle loaded drug could completely regress the cancer in rat.

Dosage Schedule:

Doxorubicin-lactoferrin nano particle (2 microgram per gram in i.v. route, 4 microgram per gram through oral route) per dose. Drug formulation is administered weekly one dose. Rats were treated for 5 weeks through i.v. and ten weeks through oral administration in the referred in Examples 8 and 9.

Effective Dose—

0.4 mg (in i.v.)/0.8 mg (in oral) of protein-doxorubicine nano particles referred in Examples 8 and 9, contain 200 microgram (in i.v.)/400 microgram (in oral) of a doxorubicin containing particles is dispersed in 0.2 ml of PBS or TBS to 125-150 gm Wistar rat.
H) Analysis of HIV-1 neutralization

SUP-T1 cells (0.4×10⁶/ml) with 100% viability were seeded in RPMI 1640, 0.1% FBS on four 12-well plates. Increasing concentration of lactoferrin nano-drug, sol-drug were added to the cells which were then infected with HIV-1_{93IN101} at a final virus concentration equivalent to 1 ng of p24 per ml. The infected cells were incubated for 2 h at 37° C. in a 5% CO₂ incubator. The cells were then pelleted at 350×g for 10 min, the supernatant was discarded, and cells were washed with RPMI 1640 containing 10% FBS. The cells were resuspended in fresh complete medium and were incubated for a further 96 h. The supernatants were then collected and analyzed using a p24 antigen capture assay kit (Advanced Bioscience Laboratories, Kensington, Md., USA). The extent of infection in the absence of test compound was considered to be equivalent to 0% inhibition.

1-13. (canceled)

14. Nanoparticles comprising lactoferrin and at least one additional component, wherein the lactoferrin has a diameter of 40 to 60 nanometers.

15. The nanoparticles of claim 14, wherein the at least one additional component is selected from the group consisting of antibiotics, anti-cancer agents, neuroactive agents, proteins, antibodies, anti-HIV agents, and DNA.

16. The nanoparticles of claim 15, wherein the antibiotic is one of cefuroxime and chloroquine.

17. The nanoparticles of claim 15, wherein the anti-cancer agent is selected from the group consisting of cyclophosphamide, ifosfamide, paclitaxel, methotrexate, fluorouracil, doxorubicin, daunorubicin, cisplatin, carboplatin, etoposide, cytarabine, gemcitabine, and docetaxel.

18. The nanoparticles of claim 15, wherein the anti-HIV agent is one of azidothymidine and enfuvirtide.

19. A method of preparing lactoferrin-containing nanoparticles comprising:
dissolving lactoferrin and at least one additional component in a solvent to produce a solution, wherein the solvent is selected from the group consisting of phosphate-buffered saline, Tris buffer, saline, and water;
dispersing the solution in an oil at a temperature of from about 4 degrees Celsius to about 30 degrees Celsius to produce a second solution;
sonicating the second solution in a sonicator in two-second pulses separated by two second periods of no pulses continuously for 15 minutes to produce a sonicated solution;
freezing the sonicated solution for at least 15 minutes at below −20 degrees Celsius, followed by incubating the sonicated solution at between about 4 degrees Celsius and about 30 degrees Celsius for 2 to 8 hours to produce an incubated solution;
centrifuging the incubated solution for 10 to 30 minutes, thereby separating lactoferrin-containing nanoparticles from supernatant;
discarding the supernatant and washing the lactoferrin-containing nanoparticles at least once with one of diethyl ether or an oil-miscible solvent; and
dispersing the lactoferrin-containing nanoparticles in one of saline, a buffer, or water.

20. The method of claim 19, wherein the ratio of the solution to oil in the dispersing step is at least 1:35.

21. The method of claim 19, wherein the oil is selected from the group consisting of olive oil, vegetable oil, castor oil, and peanut oil.

22. The method of claim 19, wherein the at least one additional component is selected from the group consisting of antibiotics, anti-cancer agents, neuroactive agents, proteins, antibodies, anti-HIV agents, and DNA

23. The method of claim 19, wherein the sonicated solution is frozen immediately after sonication and the centrifugation is carried out at at least 5000 rpm.

24. The method of claim 19, wherein the lactoferrin-containing nanoparticles are dispersed in mannitol and are lyophilized to produce a powder.

25. A method of preparing lactoferrin-containing nanoparticles comprising:
dissolving lactoferrin in a solvent to produce a solution;
dispersing the solution in oil to produce a second solution;
sonicating the second solution to produce a sonicated solution;
freezing the sonicated solution followed by incubating the sonicated solution to produce an incubated solution;
centrifuging the incubated solution, thereby separating lactoferrin-containing nanoparticles from supernatant;
washing the lactoferrin-containing nanoparticles at least once; and
dispersing the lactoferrin-containing nanoparticles in one of saline, a buffer, or water.
